(19) United States
(12) Patent Application Publication
(10) Pub. No.: US 2020/0138870 A1
(43) Pub. Date: May 7, 2020

(54) RECREATION OF PANCREATIC NICHE ALLOWS FOR NOVEL METHODS FOR HUMAN, MATURE BETA DERIVATION FROM PLURIPOTENT STEM CELLS

(71) Applicant: Baylor College of Medicine, Houston, TX (US)

(72) Inventors: Malgorzata Borowiak, Houston, TX (US); Jolanta Chmielowiec, Houston, TX (US); Diane Yang, Houston, TX (US)

(21) Appl. No.: 16/349,473

(22) PCT Filed: Nov. 16, 2017
(86) PCT No.: PCT/US2017/062097

§ 371 Date: May 13, 2019

Related U.S. Application Data
(60) Provisional application No. 62/423,471, filed on Nov. 17, 2016.

(51) Int. Cl. A61K 35/39; C12N 5/071

(52) U.S. Cl. CPC A61K 35/39; C12N 5/0676; C12N 2502/1388; C12N 2501/415; C12N 2506/02

(57) ABSTRACT

Embodiments of the disclosure concern methods and compositions related to treatment of diabetes and/or related conditions using cell therapy. In specific embodiments, cells that lacked the ability to produce insulin are exposed to one or more particular agents that render the cells to have the ability to produce insulin, and these cells are provided to an individual in need thereof. In a specific embodiment, the agent(s) are Wnt5a, FGF7, WNT3a, HGF, THBS2, IGF1, PDPN, LIF, endocan, SERPINF1, EGF, or a combination thereof.

### CROSS-REFERENCE TO RELATED APPLICATIONS

This application claims priority to U.S. Provisional Patent Application Ser. No. 62/423,471, filed Nov. 17, 2016, which is incorporated by reference herein in its entirety.

### STATEMENT REGARDING FEDERALLY SPONSORED RESEARCH OR DEVELOPMENT

This invention was made with government support under P30-DK079638 awarded by National Institutes of Health. The government has certain rights in the invention.

### TECHNICAL FIELD

Embodiments of the disclosure concern at least the fields of cell biology, molecular biology, endocrinology, biochemistry, and medicine.

### BACKGROUND

P cells are the predominant endocrine cell type of the pancreas and the only cell in the body that can generate and secrete insulin (INS) to maintain blood glucose homeostasis. Loss of functional INS-producing 3 cells causes diabetes, and the inability of available therapies to adequately stabilize blood glucose levels (Cryer, 2014; Home et al., 2014; Zaykov et al., 2016) means that even treated diabetics often develop retinopathy, neuropathy, and stroke, among other complications (Forbes and Cooper, 2013; Olokoba et al., 2012). The ideal therapy would be to replace the dysfunctional cells, and in fact, cadaveric islets can reconstitute 3 cells and restore normoglycemia. Unfortunately, each transplant requires billions of cells, and there are not enough cadaveric islets to treat the millions of people around the world with insulin-dependent diabetes. There has thus been enormous interest in regenerative medicine approaches for the treatment of diabetes.

In vitro-derived human β cells can alleviate hyperglycemia in mice (Kroon et al., 2008; Pagliuca et al., 2014; Rezania et al., 2014; Vegas et al., 2016), but human pluripotent stem cells (hPSCs) cannot yet be reliably coaxed into functional 3 cells in sufficient numbers to serve as therapy (Kroon et al., 2008; Pagliuca et al., 2014; Rezania et al., 2014; Vegas et al., 2016). The efficiency of hPSC differentiation to β cells remains low, varying from 10-30%, and the duration (4 to 5 weeks) and cost of production so far are impractical (D'Amour et al., 2006; Nostro et al., 2015; Pagliuca et al., 2014; Rezania et al., 2014; Russ and Hebrok, 2014; Russ et al., 2016). The biggest problem, however, is that in vitro-generated β cells do not behave like their mature human counterparts. Whereas mature human 3 cells secrete only INS and respond with great precision to different amounts of glucose in the blood, hESC-derived 3 cells often co-express other endocrine hormones such as glucagon and somatostatin and do not respond adequately to glucose levels. We do not know enough about how the human pancreas develops in vivo endocrine progenitors into cells that can be constantly challenged by, and responsive to glucose yet to reliably generate functional 3 cells in vitro.

Differentiation protocols commonly aim to mimic in vivo development in an in vitro system, but in vitro systems lack the neighboring cell types present in vivo. When progenitors differentiate into endocrine cells, they delaminate from an epithelial layer into the surrounding mesenchyme and thus associate closely with the pancreatic niche. Initial work in Xenopus and mouse demonstrated that endothelial cells are essential in pancreatic development (Lammert et al., 2001) and specifically for the induction of the transcription factors Pdx1 and Ptf1a, which are responsible for the formation of the organ (Jacquemin et al., 2006; Lammert et al., 2001; Yoshitomi and Zaret, 2004). Over a decade ago, Bhushan and colleagues demonstrated that fibroblast growth factor 10 (FGF10), expressed by the mesenchyme, stimulates proliferation of Pdx1+ progenitors (Bhushan et al., 2001). Since then, zebrafish and mouse studies have identified a number of signaling pathways such as retinoic acid, FGF, BMP and TGF that are important for pancreatic development (Dichmann et al., 2003; Kobberup et al., 2010; Martin et al., 2005). These interactions are temporally regulated, as blood vessels at later developmental stages restrict the outgrowth and morphogenesis of the pancreatic epithelium in mice (Magenheim et al., 2011). Irrespective of species, mature islets are highly vascularized. hPSC-derived pancreatic progenitors (PPs) are supported by ingrowing host blood vessels after transplantation. It is worth noting that transplanted islets in human patients or transplanted 3 cells in mice perdure, but survive only a short period of time in in vitro cultures. These studies suggest that something present in vivo is missing in vitro, and that identifying the signals from the surrounding niche that support the differentiation and maturation of human 3 cells could provide the missing link for the development of cell-based therapies (Negi et al., 2012).

Previously, the inventors and others demonstrated that pancreatic stage-specific mesenchyme is a source of signals that allow massive in vitro expansion of hPSC-derived definitive endoderm (DE) (Cheng et al., 2012; Sneddon et al., 2012). The role of the niche, defined here as mesenchymal and endothelial cells, at later stages, such as when human endocrine cells are developing, is not understood. It was considered that: i) interactions between human endocrine progenitors (EPs) and the pancreatic niche promote the differentiation and maturation of these progenitors into β cells, and ii) components of the human pancreatic niche change over time and differ in terms of their capability to promote β cell specification. The inventors therefore used a two-pronged strategy to identify the effect of niche on endocrine differentiation, as well as the molecular signals that collectively promote β cell specification.

First, the inventors established various human fetal pancreatic niche primary cells, comprised of mesenchymal and endothelial (M-E) cells at different stages of development, to delineate their contribution to differentiating β cells. Once the stages were identified that most strongly stimulated β cell development, the mesenchymal and endothelial signals that promote INS expression and β cell specification were then identified. Finally, it was determined that the interplay between the WNT5A/JNK and BMP signaling pathways is crucial to β cell specification and the in vitro development of functional INS-producing β cells.

The present disclosure provides a solution to long-felt need in the art to provide effective insulin-producing cells to individuals with diabetes.

### BRIEF SUMMARY

The present disclosure is directed to methods and compositions related at least to the treatment of diabetes (including type 1, type 2, and gestational diabetes), diabetes-related conditions, and pre-diabetes. The disclosure concerns cell therapy for treating diabetes of any kind and its related conditions. In particular embodiments, cells are exposed to one or more factors that may or may not be endogenous to the cells such that the exposure causes the cells to produce insulin. In one embodiment the cells are exposed to certain types and amounts of one or more factors such that the exposure mimics development of β cell differentiation in vivo. In certain embodiments, effective amounts of the insulin-producing cells are provided to an individual with diabetes, diabetes-related conditions, or pre-diabetes, for example.

Particular embodiments of the disclosure concern one or more pancreatic niche-derived factors for human endocrine development. In specific embodiments, a human pancreatic niche promotes β cell differentiation via WNT5A/JNK/AP1 and BMP signaling and at least some of the agents in the pathways therein are provided to cells to cause them to become insulin-producing.

In one embodiment, there are methods of treating an individual (infant, child, adolescent, or adult) for diabetes (type I or type II), one or more diabetes-related conditions, or pre-diabetes, comprising the step of administering to the individual an effective amount of insulin-producing cells produced upon exposure of insulin-lacking cells to one or more agents, wherein the one or more agents are selected from the group consisting of Wnt5a, FGF7, WNT3a, HGF, THBS2, IGF1, PDPN, LIF, endocan, SERPINF1, EGF, and a combination thereof. The insulin-lacking cells may be stem cells, pluripotent cells, induced pluripotent stem cells, or a mixture thereof. In particular cases, the insulin-lacking cells are embryonic stem cells. The cells may be autologous or allogeneic to the individual. In some cases, the methods include the step of obtaining the insulin-lacking cells from the individual to be treated or another individual.

In specific embodiments, the cells are administered to the individual by injection. The cells that are administered to the individual may be encapsulated. The cells that are injected may be injected into a portal vein, such as one connecting the liver and the pancreas. The cells may be administered to an individual in an encapsulation device. The cells may be administered to the individual in arginate bubbles. In certain embodiments, the cells are administered to the individual more than once. In some cases, the insulin-producing cells or insulin-lacking cells are engineered to produce one or more non-endogenous gene products. In specific embodiments, one or more cell surface receptors in the cells are modified to avoid immune system recognition of the cells.

In particular embodiments, the one or more agents comprise, consist of, or consist essentially of Endocan, SERPINF1, WNT5A, HGF, and a combination thereof. The one or more agents may comprise, consist of, or consist essentially of Endocan and SERPINF1. The one or more agents may comprise, consist of, or consist essentially of Endocan and WNT5A. The one or more agents may comprise, consist of, or consist essentially of Endocan and HGF. The one or more agents may comprise, consist of, or consist essentially of SERPINF1 and WNT5A. The one or more agents may comprise, consist of, or consist essentially of SERPINF1 and HGF. The one or more agents may comprise, consist of, or consist essentially of WNT5A and HGF. The one or more agents may comprise, consist of, or consist essentially of Endocan and SERPINF1 and WNT5A. The one or more agents may comprise, consist of, or consist essentially of Endocan and SERPINF1 and HGF.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIGS. 1A-1G show that human β cells generated in vitro in coculture with organ- and stage-specific mesenchyme-epithelial (M-E) cells secrete INS in response to low and high glucose levels. FIG. 1A) Human M-E primary cells were de novo derived from different organs and developmental stages. Previously established cell lines representing mesenchyme and endothelium were used as controls. FIG. 1B) Representative images of derived primary cells immunofluorescently stained for mesenchymal marker, Vimentin (upper panel, green) or endothelial markers, PECAM1 (lower panel, red) and CFIII (green). Nuclei are labeled with Dapi in blue. Scale bar=100 μm. FIG. 1C) Top panel: overview of main pancreatic differentiation protocol used to derive pancreatic progenitors (PPs) or endocrine progenitors (EPs) from hESCs with representative images (bottom panel) of definitive endoderm (DE) stained for SOX17, PPs for PDX1 (green) and NKX6.1 (red), and EPs stained for NGN3 (red). Nuclei are visualized with Dapi, blue. Scale bar=250 μm. FIG. 1D) Overview of coculture approach to induce β cells from PPs. hESCs are differentiated into PPs in vitro and cultured on M-E cells for 3, 7, or 14 days, during which cells are characterized by immunofluorescence (IF), qPCR, and FACS. FIG. 1E) Representative images of β cells probed with C-peptide antibody after 7-days cocultured with Wk17.5, Wk9.1, HUVECs, or mefs. Nuclei are visualized with Dapi in blue. Scale bar=100 μm. FIG. 1F) Whisker plot quantification (on the right) of C-peptide+ cells as fold change normalized to result without (W/O) coculture. Data are represented as mean±SD. The middle horizontal line indicates the median. n=5 biological replicates. Significance was determined using one-way analysis of variance (ANOVA) with Dunnett's multiple comparisons test. Mean of each column was compared with mean of control column (without coculture). ** p<0.01, ***p<0.001. FIG. 1G) Representative FACS plot of INS+ and GCG+ cells after 7-days cocultured with Wk17.5h M-E cells.

FIGS. 2A-2F demonstrate that human β cells induced by M-E cells are glucose-responsive in vitro, and ECM or conditional media from M-E cells increases INS-positive cells in pancreatic progenitors. FIG. 2A) GSIS analysis at day 3, 7, and 14 after pancreatic progenitor (PP) coculture with M-E lines. Graph representing ELISA measurements of human C-peptide from normalized cells after stimulating with 2.8 mM and 16.7 mM glucose at day 3, 7, or 14 of coculture with M-E and control cells. n=8 biological replicates and 3 technical replicates. Results were normalized to cell number and total protein content. A′) GSIS at day 3 of coculture. Cells were challenged with 2.8 mM and 16.7 mM glucose. After low/high glucose stimulation, cells were depolarized with 30 mM KCl and secreted human C-peptide was measured by ELISA (Mercodia). Results were normalized to cell number and total protein content. A″) GSIS at day 14 of coculture as described above, except different range of Y-axis scale. n=8 biological replicates and 3 technical replicates. A′″) Cells were challenged with 2.8 mM and 16.7 mM glucose. After low/high glucose stimulation, cells were depolarized with 30 mM KCl. Stimulation index for cells from coculture at day 3, 7, and 14 was calculated as a ratio between 16.7 to 2.8 mM glucose stimulation (lower panel, on the right). n=8 biological replicates for all groups except of human islets, n=4, and 3 technical replicates for all groups. FIG. 2B) Schematic overview of approach to determine the contribution of ECM matrix from M-E cells to PP differentiation into β cells. FIG. 2C) Schematic overview of approach to determine the contribution of conditional media from M-E cells to PP differentiation into β cells. FIG. 2D) Immunofluorescent analysis of INS+ cells (red) after PPs cultured on the ECM matrix for 14 days are presented in the upper panel and of C-peptide+ cells (red) after PPs cultured in conditional media (CM) for 14 days in the lower panel. Nuclei are labeled with Dapi shown in blue. Scale bar=100 μm. FIG. 2E) Whisker plot quantification of C-peptide+ cells in each conditional media experiment as fold change over basic media without coculture. Data are represented as mean±SEM. The middle horizontal line indicates the median. n=5 biological replicates. Significance was determined using one-way analysis of variance (ANOVA) with Dunnett's multiple comparisons test. Mean of each column was compared with mean of control column (without coculture). *p<0.05, ** p<0.01. FIG. 2F) Overview of approach to determine when Wk17.5 or Wk20.1 primary M-E cell coculture potentiates C-peptide+ cell induction is shown in the upper panel with the quantification of C-peptide+ cells after either PPs or EPs were cocultured with primary M-E cells. Data are presented as mean fold change normalized to non-coculture control ±SEM.

FIGS. 3A-3G show that human Wk17.5h and 20.1 pancreatic niche exhibit unique signatures with conserved ECM components and secreted factors. FIG. 3A) Primary M-E cells from Wk17.5 and Wk20.1 are similar yet not identical. Comparison of the log 2 gene expression levels between Wk17.5h and 20.1 M-E lines, with moderate correlation (R=0.7878). Selected conserved secreted factors and ECM components are annotated in blue. FIG. 3B) Human niche cells are distinct from other mesenchymal cell line. Venn diagram of genes from human fetal Wk17.5h (blue) and Wk20.1 (yellow) M-E primary cells significantly changed compared to control HDF cells. FIG. 3C) Functional gene annotation of significantly enriched pathways and processes of unique genes upregulated in Wk17.5h (FC>20, p<0.05 top panel) and heatmap of genes associated with top upregulated process by gene ontology: cell communication. FIG. 3D) Functional gene annotation of significantly enriched pathways and processes of unique genes upregulated in Wk20.1 (FC>20, p<0.05, top panel) and heatmap of genes associated with top upregulated process by gene ontology: cell receptor signaling, adhesion and ECM regulation. FIG. 3E) Functional gene annotation of significantly enriched pathways and processes among upregulated genes shared by Wk17.5h and Wk20.1 M-E cells (FC>20, p<0.05). FIG. 3F) Molecular pathway analysis of secreted factors and ECM components from upregulated genes shared by Wk17.5h and Wk20.1 M-E (FC>20, p<0.05) executed by Panther GO analysis. FIG. 3G) Heatmap of ECM and secreted growth factors enriched in human Wk17.5 and 20.1 pancreatic niche compared to HDFs. Log₂ Z-score of normalized expression values of ECM and secreted growth factors as determined through GO analysis.

FIGS. 4A-4F show that selected growth factors secreted from M-E cells differentiate hESC-derived endocrine progenitors into CHGA- and INS-positive cells. FIG. 4A) Experimental design: hESCs were differentiated into endocrine progenitors (EPs) and then incubated with media only, or with individual growth factors: FGF7, HGF, PDPN, SERPINF1, WNT5A, WNT5B, EGF, THBS, IGF, Endocan, LIF, or WNT3A for 3 days followed by immunofluorescence analysis. FIG. 4B) INS (red) immunofluorescence staining after Endocan, SERPINF1, WNT5A, HGF, and FGF7 treatment are shown as examples. Nuclei are stained with Dapi (blue). Images of high concentration treatment (Table 3) are shown. Scale bar=100 μm. FIG. 4C) INS+ cells out of total (Dapi+) cells are presented as mean±SEM, n=5 independent biological replicates. Significance was determined using one-way analysis of variance (ANOVA) with Bonferroni's multiple comparisons test *p<0.05, ** p<0.01, ***p<0.001. FIG. 4D) CHGA+ out of total (Dapi+) cells are presented as mean±SEM, n=5 independent biological replicates. Significance was determined using one-way analysis of variance (ANOVA) with Bonferroni's multiple comparisons test *p<0.05, ** p<0.01, ***p<0.001. FIG. 4E) WNT5A facilitated differentiation of EPs generated by independent protocol in 3D approach. ISL1-EGFP hESCs were differentiated as 3D organoids (Pagliuca et al., 2014) until EP stage, where WNT5A was added for 2 days together with T3, ALK5i in CMRL media. INS (red) was increased as early as at day 4 after WNT5A treatment. Nuclei are shown in blue by Dapi. At day 12, the number of INS+SC-β cells corresponds to the number of INS+ in WNT5A-treated cells at day 4. Scale bar=100 μm. FIG. 4F) Quantification of INS+ cells out of total (Dapi+) cells after 4 and 12 days of WNT5A treatment of 3D-EPs. Data are presented as mean±SEM, n=5 independent biological replicates. Statistical significance was determined using t-test, *p<0.05

FIGS. 5A-5N show that WNT5A is expressed in human pancreatic niche during development and promotes INS expression in vitro. FIG. 5A) Human Wk16.3 pancreas stained for WNT5A (red, left and middle panel) and PECAM1 (green, left panel) or Vimentin (green, middle panel), and CHGA (blue). Wk20.1 pancreas stained for Vimentin (green), WNT5A (blue) and CHGA (red) is shown in the right panel. Scale bar=100 μm. FIG. 5B) qPCR analysis of WNT5A expression in hESC, and their derivatives: DE, PPs, EPs and β cells. *p<0.05, ***p<0.001, t-test. FIG. 5C) Flow cytometry analysis of WNT5A expression in hESC-derived EPs and β cells co-stained for CHGA and INS, respectively. FIG. 5D) WNT5A is expressed in subpopulations of human naïve adult β cells and its expression is lost in diabetic patients. Single cell transcriptional analysis of WNT5A and INS expression from normal non-diabetic (left panel) and type 2 diabetic (T2D) (right panel) adult β cells from Lawlor et al., 2016. Each dot represents log 2 (CPM) expression of individual cells from 168 normal and 96 T2D cells. FIG. 5E) Human islet stained for WNT5A (red, left panel) and INS (green, middle panel) and merged image (right panel). Scale bar as 100 μm. FIG. 5F) Human islet stained for WNT5A (blue), SST (green), and GCG (red). F′) Human islet stained with INS (green) and FZD3 (red) antibodies. Scale bar as 100 μm. FIG. 5G) Human islet stained for FZD3 (red, left panel) and WNT5A (green, middle panel) and merged image (right panel) with nuclei marked by Dapi (blue). FIG. 5H) hESC-derived EPs stained for FZD3 (red) and PDX1 (green). Nuclei are stained with Dapi (blue). Scale bar as 100 μm. H′) hESC-derived β cells stained for FZD3 (red) and INS (green). Nuclei are stained with Dapi (blue). Scale bar as 100 μm. FIG. 5I) WNT5A is lost in Wk20.1 WNT5A KO M-E cells as determined by immunofluorescence. Nuclei are stained with Dapi (blue). Scale bar=100 μm. FIG. 5J) Wk17.5h WNT5A KO M-E cells were cocultured with EPs for 4 days and INS+ cells were evaluated and compared to control EPs (no coculture). Data are presented as mean±SEM, n=5, ***p<0.001, t-test. FIG. 5K) HDFs were nucleofected with pCDNA3.0 or pCDNA-WNT5A (pW5A), and cocultured with EPs. After 3 days INS+ cells were quantified. Data are presented as mean±SEM, n=3, *p<0.05, t-test. FIG. 5L) hESC-derived EPs, after ectopic WNT5A expression (pWNT5A), were stained for WNT5A (green, top panel) and INS (red, bottom panel). Nuclei are labeled with Dapi, in blue. Backbone plasmid was used as mock control. Scale bar=100 μm. FIG. 5M) Fold change of INS+ cells after ectopic WNT5A expression (pWNT5A) in EPs normalized to EPs transfected with backbone plasmid is shown. Data are presented as mean±SEM, n=3, ***p<0.001, t-test. FIG. 5N) hESC-derived EPs treated with 1 μg of WNTSA neutralizing antibodies for 3 days. INS+ cells out of total (Dapi+, blue) cells was quantified using immunofluorescence and are presented as fold change normalized to IgG treated control. Data are presented as mean±SEM, n=3, **p<0.01, t-test.

FIGS. 6A-6H demonstrate that short-term WNTSA treatment activates JNK/c-Jun pathway in human EPs. FIG. 6A) Experimental design of global gene expression changes in human EPs induced by short- (12h) and long-term (5 days) WNTSA treatment by RNA-seq. FIG. 6B) WNTSA treatment shifts hESC-derived EPs towards the transcriptional profile of 3 cells. Z-score of normalized RNA-seq expression values of selected genes with at least one pairwise difference of q<0.05. FIG. 6C) qPCR verification of selected RNA-seq results. Expression of INS, GCG, CHGA, ONECUT1, and PCSK2 were evaluated in 5 days untreated (−) and WNT5A-treated EPs. FIG. 6D) Predicted significantly upregulated TFs in EPs after short-term WNT5A treatment indicating high c-JUN upregulation as analyzed by TFactS. FIG. 6E) Short-term (12h) WNT5A treatment activates JNK and it leads to increase in total JNK expression and phosphorylation, as demonstrated by Western Blot for p-JNK, JNK, and beta actin as loading control. FIG. 6F) JNK inhibition by small molecule antagonist (SP600125) lowers the INS+ cell induction in EPs. EPs were treated for 3 days with SP600125 at two concentrations and INS+ were quantified and presented as fold changed compared to vehicle (DMSO) treated control. Data are presented as mean±SEM, n=5, ***p<0.001, t-test. FIG. 6G) p-c-JUN (green) and INS (red) staining in EPs treated for 24h with WNT5A or in untreated (UT) control. Nuclei are visualized with Dapi (blue). Scale bar=100 μm. FIG. 6H) Quantification of p-c-JUN+ cells shown are percentage out of total (Dapi+) cells in untreated (UT) and WNT5A treated EPs showing 6 fold increased in number of c-JUN+ cells. Data are presented as mean±SEM, n=6, **p<0.01, t-test

FIGS. 7A-7K. demonstrate that long-term WNT5A treatment inhibits BMP signaling in hESC-derived EPs. FIG. 7A) Gene expression changes in BMP pathway components, including downregulation of BMP ligands and effectors but upregulation of BMP antagonists, indicate the decrease in BMP activity after long-term (5d) WNT5A treatment. Log₂ Z-score of normalized RNA-seq expression values of BMP pathway related genes are represented as a heatmap. FIG. 7B) qPCR verification of selected genes from the BMP pathway expression after 5 day-WNT5A treatment. WNT5A EP treatment leads to downregulation of BMP3, BMP4 and BMP6 and upregulation of the BMP inhibitor, BMPER. Data are presented as mean±SEM, n=6, *p<0.05, t-test. FIG. 7C) C1. Multigenic construct used in the dual-pathway luciferase assay. All elements are included in the same DNA string ensuring the simultaneous transfection of all the reporters in equal ratios in the cells. 4 copies of the Smad binding element (4×Smad_RE) were cloned upstream of a synthetic minimal TATA-box promoter with low basal activity (miniP) to drive the expression of the Red Firefly luciferase (RedF). 6 copies of the AP-1 binding element (6×AP1) were assembled upstream of the miniP to drive the expression of the Firefly luciferase (FLuc). The expression of the standard luciferase Renilla was driven by the Cytomegalovirus enhancer and promoter. Each transcriptional unit included the bovine growth hormone terminator (bGHT) and a synthetic polyA -p(A)n- and a transcriptional pause signal—Pause—were added upstream of the DNA response elements to prevent interference derived from the transcription of the upstream luciferase. (C2) Recorded spectra of Firefly (FLuc) and Red Firefly (RedF). The 530-40 band pass filter (BP) used for the luciferase measurement is indicated over the spectra. (C3) The transmission constants for each luciferase (KFLuc530 and KRedF530) were calculated by dividing the transmitted light (FLuc530 and RedF530) by the total light emitted by each luciferase (FLucTOTAL and RedFTOTAL). (C4) Simmultaneous equation for calculating luciferase activity in the Red Firelfy and Firefly Luciferase mixutre. LightTOTAL is the total relative light units (RLU) measured in the absence of the optical filter, FLuc530 are the RLU of FLuc that pass though the BP, RedF530 are the RLU that pass though the 530-540 BP and FLuc and RLuc are the Firefly luciferase and the Red firefly luciferase contribution to the mix, respectively. (C5) Overview of luciferase assay; three luciferase measurements are performed, two at 2 seconds after LARII reagent injection and the third one at 4 seconds after Stop & Glo reagent injection. FIG. 7D) Quantification of relative AP1 and Smad activity in EPs treated for different time length with WNT5A showing first increased AP1 transcriptional activity followed by a decrease in Smad activity. Anisomycin (Anis) and BMP4 were used as positive control for AP1 and Smad, respectively. Data are presented as mean±SEM, n=3 biological replicates. Statistical significance was determined by one-way analysis of variance (ANOVA) with Bonferroni's multiple comparisons test. *p<0.05, ** p<0.01. FIG. 7E) Immunofluorescent analysis to determine cellular localization of phosphorylated Smad1 and 5 in INS+ cells. p-Smad1/5 is mostly localized in cytoplasm (CYT) in cells with high INS expression, while it is present in nucleus (NC) in INS− cells, suggesting correlation between Smad activity and INS expression. FIG. 7F) Quantification of cytoplasmic and nuclear p-Smad1/5 localization in INS+ and INS− cells showing that 77% cells correlate INS with cytosolic p-Smad1/5 localization. Data are presented as mean±SEM, n=3 biological replicates, at least 10 images were quantified. Significance was determined by t-test, ***p<0.001. FIG. 7G) Representative images from imaging flow cytometry of single EP cell stained for pSmad1/5 (green), INS (yellow), and Dapi (purple). Vast majority of cells expressing nuclear pSmad1/5 (top panel) did not express INS, and cells with cytosolic pSmad1/5 (bottom panel) show strong INS expression. FIG. 7H) Fold change of CHGA+ and INS+ cell number compared to untreated cells quantified by immunofluorescence staining after 3 days treatment of 50 or 200 ng/ml Gremlin1 (Grem), 100 or 500 ng/ml WNT5A or in combinations. Data are presented as mean±SEM, n=5 biological replicates. Significance was determined using one-way analysis of variance (ANOVA) with Bonferroni's multiple comparisons test. *p<0.05, ** p<0.01, ***p<0.001. FIG. 7I) INS immunostaining (red) of hESC-derived EPs treated for 3 days with 500 ng/ml of WNT5A, 200 ng/ml Gremlin1, and Gremlin1 with WNT5A together. Nuclei are stained with Dapi (in blue). Scale bar=100 μm. FIG. 7J) GCG+ cells were evaluated after 3-day EP treatment with WNT5A, Gremlin1, or in combination by immunofluorescence and the fold change in GCG+ cell number as compared to untreated control (UT) is shown. Data are presented mean±SEM, n=4 biological replicates. Significance was determined using one-way analysis of variance (ANOVA) with Bonferroni's multiple comparisons test *p<0.05, ***p<0.001. FIG. 7K) Proposed model of WNT5A role in pancreatic niche during human EP to β cell differentiation. Pancreatic stage specific M-E cells secrete WNT5a and Gremlin1, and these growth factors cooperate to activate JNK/c-Jun/AP1 signaling while inhibiting the BMP pathway which in turn leads to upregulation of CHGA, INS, and downregulation of GCG, in hESC-derived EPs.

FIGS. 8A-8F show that pancreatic niche-derived primary cells express mesenchymal and endothelial markers but not epithelial. FIG. 8A) and FIG. 8B) Characterization of de novo derived pancreatic niche cell lines by qPCR analysis of mesenchymal (VIMENTIN, FSP1) and FIG. 8B) endothelial marker (PECAM1, FLK1, VE CADHERIN, ICAM, VWF) expressions in HUVECs, HDFs, Wk9.1, 17.5h and 20.1 lines. Gene expression was normalized to TBP. Data are presented as mean±standard error from 3 independent experiments. FIG. 8C) VIMENTIN expression at passage 25 in control mesenchymal cells, HDFs and PECAM1 expression at passage 8 in control two endothelial cell lines, HUVECs and MS1. Data are presented as mean±standard error from 3 independent experiments. FIGS. 8D-8F) Mesenchymal and endothelial cells do not express pancreatic islet cell markers. qPCR of INS and PDX1 expression or FOXA2 and SOX9 expression in Wk9.1, 17.5h, 20.1 and hESCderived β cells (iBeta cell) or hESC-PPs. Data are presented as mean±standard error from 3 independent experiments.

FIGS. 9A-9B shows glucose stimulated insulin secretion (GSIS). FIG. 9A) GSIS at day 7 of coculture. Cells were challenged with 2.8 mM and 16.7 mM glucose. After low/high glucose stimulation, cells were depolarized with 30 mM KCl and secreted human Cpeptide was measured by ELISA (Mercodia). Results were normalized to cell number and total protein content. FIG. 9B) INS+(red) cells induced by coculture with Wk20.1 M-E cells co-express NKX6.1 (green). Cell nuclei are stained by Dapi (in blue).

FIGS. 10A-10G. Pancreatic niche-derived M-E cells signals promote β cell development. FIG. 10A) CHGA (red) immunofluorescence staining after Endocan, SERPINF1, WNT5A, HGF, and FGF7 treatment are shown as examples. Nuclei are stained with Dapi (blue). Images of high concentration treatment (Table 3) are shown. Scale bar=100 μm. FIG. 10B) ISL1-EGFP cells stained with GFP antibody after untreated (B27 media only) control, Endocan, SERPINF1, WNT5A, HGF, and FGF7 treatment. Each growth factor was used at two concentrations. FIG. 10C) Quantification of ISL1-EGFP+ cells after growth factor treatment for 3 days. Numbers of positive cell were normalized to B27 control. Data are presented as mean±standard error from 3 independent experiments. Statistical significance was evaluated with ANOVA one-way with Dunnett's multiple comparisons test (*p<0.05, ** p<0.01, *** p<0.001). FIG. 10D) Pancreatic niche-derived growth factors induce C-peptide expression in EPs. Quantification of C-peptide+ out of total Cells (Dapi+) after 3-day incubation of EPs with B27 control, or Endocan, SERPINF1, WNT5A, HGF, and FGF7. Data are presented as mean±standard error from 5 independent experiments. Statistical significance was evaluated with ANOVA one-way with Dunnett's multiple comparisons test (*p<0.05, ** p<0.01, *** p<0.001). FIG. 10E) Quantification of INS+ cells induced from H1 hESC-derived EPs after growth factor treatment for 3 days. Data are presented as mean±standard error from 3 independent experiments. Statistical significance was evaluated with ANOVA one-way with Dunnett's multiple comparisons test (*p<0.05, ** p<0.01, *** p<0.001). FIG. 10F) INS (in red) immunostainings after Endocan (E), SERPINF1 (S), WNT5A (W), and HGF (H) combinational treatment. Images for high concentration treatment are shown. Scale bar=100 μm. FIG. 10G) Quantification of INS+ cells of combinational treatment of Endocan (E, in red), SERPINF1 (S, in blue), HGF (H, in orange), and WNT5A (W, in green). Data are presented as mean±standard error from 3 independent experiments. Statistical significance was evaluated with ANOVA one-way with Dunnett's multiple comparisons test (*p<0.05, ** p<0.01, *** p<0.001; color indicates comparison group). The gray background indicates treatments that most efficiently induced INS+ cells, which all contain WNT5A.

FIGS. 11A-11G show WNT5A signaling in pancreatic niche. FIG. 11A) qPCR evaluation of WNT5A expression in pancreatic primary M-E cells. FIG. 11B) qPCR validation of WNT5A expression in positive control, ovarian cancer OVCA420 cells. FIG. 11C) Validation of WNT5A antibodies using OVCA420 cell line as positive control. Immunofluorescent images of OVCA420 cells stained with WNT5A antibodies (top panel) or only secondary antibodies, (bottom panel) are shown on left. FIG. 11D) Blocking FZD3 receptor in EPs by neutralizing antibodies leads to 2.5-decrease in number of INS+ cells. Statistical significance was evaluated with Student's t-test (*p<0.05). FIG. 11E) Strategy to generate knockout WNT5A in Wk17.5 and 20.1 cells using CRISPR-Cas9 nickase system. FIG. 11F) PCR verification of WNT5A KO in Wk17.5h M-E cells. PCR was performed using genomic DNA from control Wk17.5h cells, and Wk17.5h cells targeted and antibiotic (+G418) selected (WNT5A KO). The PCR primers bind to the 3′end-targeting site. Methods and primers were described previously (Yang et al., 2016). FIG. 11G) Efficiency evaluation of WNT5A overexpression in FIGS. 5L and 5M.

FIGS. 12A-12D show that WNT5A does not act through canonical WNT signaling or increases cell migration in EPs. FIG. 12A) WNT5A treatment has modest effect in EP proliferation. EPs were stained with phospho-Histone H3 antibody (pH3, red) after 3 days of WNT5A treatment. Percentages of positive cells are shown in the bottom panel. Data are presented as mean±standard error from 3 independent experiments. Statistical significance was evaluated with Student's t-test (*p<0.05, **p<0.01, ***p<0.001). FIG. 12B) Activation of canonical WNT signaling was evaluated by TOPFLASH reporter assay. TOPFLASH (TOP) or FOPFLASH (FOP) plasmids were transfected to EPs for 48h and then cells were treated with DMSO (negative control), 100 or 500 ng/ml WNT5A, or positive control CHIR99021 (Chir) for 3 days. pRLTK, which transcribed Renilla, was cotransfectedtogether with TOPFLASH or FOPFLASH in all experimental groups as internal control. Ratio of Luciferase/Renilla is normalized to TOP/untreated group and shown as relative activity. Data are presented as mean positive cell ±SD, *p<0.05 n=3). FIG. 12C) Cell migration evaluation in EPs after WNT5A treatment. Scratches were made in mitomycin C treated EPs followed by treatment with 100 or 500 ng/ml WNT5A. Black lines indicate scratch position at 0h. Cell migrated to the gap at wound closure at 30h were quantified, as cell number in the gap/area of the gap in mm2 and results are graphed in the right panel. Data are presented as mean positive cell ±SD, n=3. Statistical significance was evaluated with Student's t-test, NS. FIG. 12D) Transwell migration and chemotaxis assay. Experimental design: the upper chambers of 8transwells were seeded with hESC-derived EPs. The bottom chambers were filled with B27 media (control), 100, 500 ng/ml WNT5A, Wk9.1, 17.5h or 20.1 conditional media. Transwell plates were incubated in 37° C. for 6 days to allow migration with media refreshed every other day (left panel). Cells attached to the bottom well were stained with Dapi and quantified (right panel). Data are shown as mean positive cell ±SD, n=3. Statistical significance was evaluated with Student's t-test, *p<0.05.

FIGS. 13A-13B show that WNT5A treatment activates JUN pathway in EPs. 13A) GSEA plot from RNA-seq data indicating that JUN pathway is positively regulated by short-term WNT5A treatment of EPs. 13B) Heatmaps showing JNK target upregulated genes in hESC-derived EPs after short-term WNT5A treatment.

FIG. 14 demonstrates that implanted in vivo WNT5A-treated hEPs efficiently differentiate into β cells. Representative immunofluorescent staining of 5-week post-transplantation graft for INS (top and middle panel, red), PDX1 (middle panel, green), C-peptide (lower panel, red) and Dapi (blue) of untreated- or WNT5A-treated EPs. Scale bar, 100 μm.

### DETAILED DESCRIPTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

The term “therapeutically effective amount” as used herein refers to that amount which, when administered to a subject or patient for treating a disease, is sufficient to effect such treatment for the disease, including to ameliorate at least one symptom of the disease.

Current efforts to differentiate pancreatic progenitors into mature human β cells as a treatment for diabetes are hindered by a lack of understanding of the conditions that promote differentiation and, especially, maturation of these cells. Here, the human pancreatic niche was analyzed at a number of timepoints (weeks 9-20) and it was found that the human niche changes from week to week and is unique in the factors that guide in vitro development of endocrine progenitors into physiologically competent β cells. Identified herein is a panel of secreted factors necessary for endocrine differentiation and it was found that WNT5A, in particular, markedly improved β cell differentiation and maturation in vitro. WNT5A initially acts through the non-canonical (JNK/c-Jun/AP1) WNT signaling pathway and later cooperates with Gremlin1 to inhibit BMP pathway, in particular embodiments. These factors can be used to mimic in vivo conditions in an in vitro system to generate bona fide β cells for translational applications.

I. Cells and Modifying Agents

In particular embodiments of the disclosure, cells that lack the ability to produce insulin are or were manipulated to produce insulin, and at least in some cases are provided to an individual in need thereof. The manipulation includes at least the following: exposure of the cells that lack the ability to produce insulin to one or more agents such as those selected from the group consisting of Wnt5a, FGF7, WNT3a, HGF, THBS2, IGF1, PDPN, LIF, endocan, SERPINF1, EGF, and a combination thereof, and following this exposure, the cells are capable of producing insulin. The exposure of insulin-lacking cells to the one or more agents occurs in vitro or ex vivo. As such, the cells are not products of nature and the methods do not occur in nature, such as either by accident or by standard biological processes.

In particular embodiments, the cells to which the one or more agents are exposed are stem cells, pluripotent cells, pluripotent stem cells, induced pluripotent stem cells, totipotent stem cells, and so forth. Any stem cells may or may not be embryonic.

The cells produced by methods herein are not naturally occurring and only exist because of manipulation by the hand of man.

In some embodiments, cells are manipulated to express insulin that prior to the manipulation would not express insulin at a detectable level. Following the manipulation (such as by exposure to one or more agents), the cells may express insulin but may not express one or more other endocrine hormones (such as glucagon and somatostatin). The insulin-producing cells following exposure to one or more agents may or may not express one or more certain beta cell transcription factors, such as Pdx1, Nkx6.1, MafA, and/or Nkx2.2). The insulin-producing cells following exposure to one or more agents may or may not express factors for glucose sensing and insulin processing or secretion, such as gluts, PC1/3, and Kir channels.

The agent(s) to which the cells are exposed so that the cells become insulin-producing are particular, and one or more of the agents may be sufficient to allow the developed ability of insulin production. In some cases, one or more agents may allow the onset of production of insulin and one or more agents used in addition to this may increase the level of insulin production in the cells.

In particular embodiments, the one or more agents include at least Wnt5a, FGF7, Wnt3a, HGF, THBS2, IGF1, PDPN, LIF, endocan, SERPINF1, EGF, or a combination thereof. In some cases Wnt5a is included in methods of producing insulin from cells that previously lacked the ability to produce insulin. In particular aspects, in addition to Wnt5a one or more other agents are utilized in the methods. The methods may utilize 1, 2, 3, 4, 5, 6, 7, 8, 9, 10, 11, or more agents to produce insulin-producing cells. Some methods may include at least Wnt5a and FGF7, at least Wnt5a and Wnt3a, Wnt5a and HGF, Wnt5a and THBS2, Wnt5a and IGF1, Wnt5a and PDPN, Wnt5a and LIF, Wnt5a and endocan, Wnt5a and SERPINF1 or Wnt5a and EGF, for example. In some embodiments, the methods include 2 or more of Wnt5a, FGF7, Wnt3a, HGF, THBS2, IGF1, PDPN, LIF, endocan, SERPINF1, or EGF.

In particular embodiments, a functionally active fragment of Wnt5a, FGF7, Wnt3a, HGF, THBS2, IGF1, PDPN, LIF, endocan, SERPINF1, and/or EGF are utilized in the methods instead of the entirety of the agent. Such a functionally active fragment may include an active site and/or particular functional domain of the agent, for example.

II. Methods of Producing the Cells

In particular embodiments, a group of cells that are not capable of producing insulin are exposed to one or more agents, and the exposure allows the cells then to produce insulin. In some cases, were it not for exposure of the one or more agents to the cells, the cells would not have been capable of producing insulin, such as in an in vitro setting.

Producing cells to make insulin includes exposure of certain cells to one or more agents. In specific embodiments, stem cells or pluripotent cells (or a mixture thereof) are exposed to one or more of Wnt5a, FGF7, Wnt3a, HGF, THBS2, IGF1, PDPN, LIF, endocan, SERPINF1, and EGF. The exposure may occur in a culture, for example.

Cells may be obtained from an individual to be treated with the cells, obtained from a different individual, or they may be obtained commercially, for example. The cells may come from a cell line. The cells may come from storage or a cell repository. The cells may or may not be obtained from a fetus, infant, child, adolescent, or adult. The cells may be obtained from the pancreas, duodenum, spleen, skin, blood or other organ. The cells may or may not be passaged prior to exposure to the one or more agents. In cases wherein the cells are exposed to one or more agents in culture, the culture media may or may not be changed during the culturing. The cells may or may not be reprogrammed to the pluripotency prior to exposure to the one or more agents. An example of reprogramming includes by transient overexpression of Oct4, Klf4, Sox2 and c-myc genes using modified mRNA and transfection; a few weeks after transfection of these genes, morphological distinct colonies are picked, expanded, and characterized regarding endogenous pluripotency markers like SSE4, Oct4, Nanog.

In some cases, when more than one agent is utilized to produce insulin-producing cells, the multiple agents may or may not be exposed to the cells at the same time. Exposure of insulin-lacking cells to the one or more agents may occur over a specific time, such as over the course of days, weeks, or months, for example. Such exposure may or may not be continual until the cells are to be utilized. In some cases, the cells are exposed to the agent(s) for 1, 2, 3, 4, 5, 6, or 7 or more days. In some cases, the cells are exposed to the agent(s) for 1, 2, 3, 4, or more weeks. In some cases, the cells are exposed to the agent(s) for 1, 2, 3, 4, 5, 6, 7, 8, 9, 10, 11, 12, or more months.

The concentration of the agent(s) to which the cells are exposed may be of any suitable amount and may be determined empirically for each agent using routine methods in the art. In some cases the concentration is at least or no more than 5, 10, 15, 20, 25, 30, 35, 40, 45, 50, 75, 100, 125, 150, 175, 200, 225, 250, 275, 300, 325, 350, 375, 400, 425, 450, 475, 500, 600, 700, 800, 900, or 1000 or more ng/ml.

III. Methods of Treatment

In embodiments of the disclosure, there are methods of treating an individual for diabetes and/or complications from diabetes or for treating pre-diabetes. Methods include administering particular cells to an individual in need thereof. The individual may have any type of diabetes, including type I, type II, Maturity-Onset Diabetes of the Young (MODY), or gestational diabetes.

In particular embodiments, there are methods of treating an individual for diabetes, diabetes-related condition, or pre-diabetes, comprising the step of providing to the individual an effective amount of cells that were exposed to one or more agents under suitable conditions for the cells to produce insulin.

In some embodiments, there are methods of treating an individual for diabetes, diabetes-related condition, or pre-diabetes, comprising the step of providing to the individual an effective amount of cells that were exposed to one or more agents under suitable conditions for the cells to produce insulin when prior to the exposure the cells did not produce insulin.

In certain embodiments, there are methods of treating an individual for diabetes, diabetes-related condition, or pre-diabetes, comprising the step of providing to the individual an effective amount of insulin-producing cells produced upon exposure to one or more agents under suitable conditions.

In some embodiments, there are methods of treating an individual for diabetes, diabetes-related condition, or pre-diabetes, comprising the step of providing to the individual an effective amount of cells previously exposed to sufficient amounts of one or more agents, wherein the cells produce insulin.

In some embodiments, there are methods of treating an individual for diabetes, diabetes-related condition, or pre-diabetes, comprising the steps of exposing cells to a sufficient amount of one or more agents such that the cells produce insulin; and providing to the individual a sufficient amount of the cells.

In certain embodiments, there are methods of treating an individual for diabetes, diabetes-related condition, or pre-diabetes, comprising the steps of exposing cells that do not produce insulin to a sufficient amount of one or more agents such that the cells produce insulin; and providing to the individual a sufficient amount of the cells.

Methods of treating the individual may or may not include the step of producing the cells that produce insulin.

In some cases, the cells to which the one or more agents are provided lack production of insulin, whereas in alternative cases the cells prior to exposure to the agent(s) may produce insulin but at an insufficient level, and then exposure of the cells to the one or more agents increases the level of endogenous insulin.

In particular embodiments, a therapeutically effective amount of the insulin-producing cells are provided to the individual in need, and the amount may be at least 1×10⁵ cells and may be up to or more than 1×10⁹ cells.

The administering of the cells to the individual may occur by any suitable method. Steps may be taken to protect the administered cells from the host immune system. In some cases the cells are injected into the individual, for example through the portal vein between the liver and pancreas. In some cases the cells are encapsulated and delivered in such a form. The cells may or may not be encapsulated in a device (as an example, the Encaptra® cell delivery system) and delivered to the individual in the device. The device may be implanted under the skin of the individual. The device may be comprised of polycaprolactone, for example.

The cells may be encompassed within microbubbles (for example, alginate microbubbles) or they may be encompassed individually.

In some cases, one or more complications from diabetes are treated with cells produced by methods of the disclosure, such as neuropathy, ketoacidosis, kidney disease, Vision loss, hypoglycemia, hyperglycemia, and so forth.

In some cases, an additional therapy to the therapy encompassed herein is given to the individual, has been given to the individual and/or will be given to the individual. Such a treatment may be of any kind, such as insulin and other injectables; healthy eating and exercise; sulfonylureas; biguanides; meglitinides; thiazolidinediones; DPP-4 inhibitors; SGLT2 inhibitors; Alpha-glucosidase inhibitors; and/or bile acid sequestrants, for example.

### EXAMPLES

The following examples are presented in order to more fully illustrate the preferred embodiments of the invention. They should in no way, however, be construed as limiting the broad scope of the invention.

### Example 1

### Human Pancreatic Niche Temporally Regulates Progenitor Differentiation into Functional B Cells

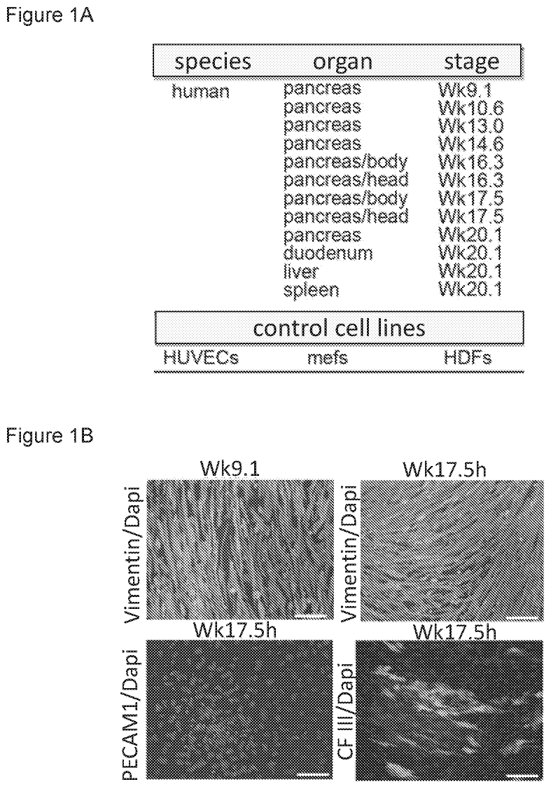

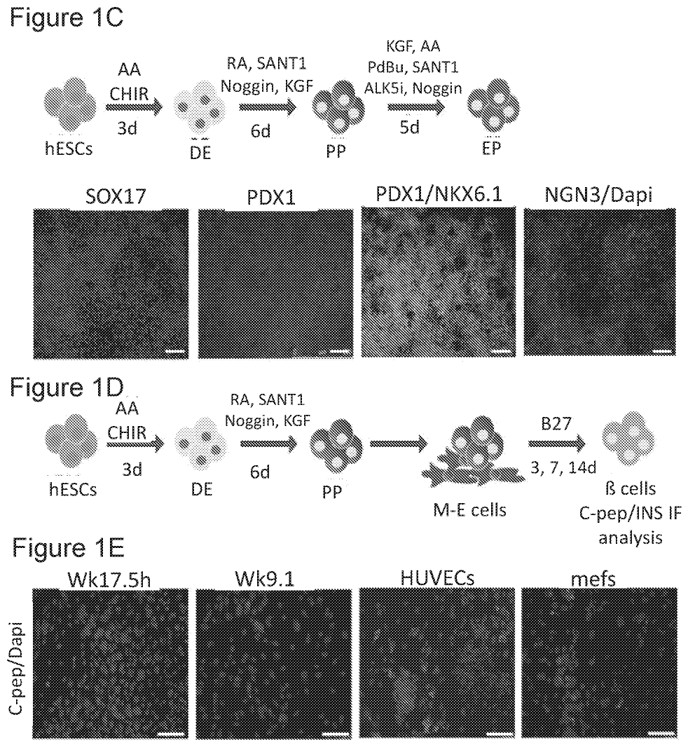

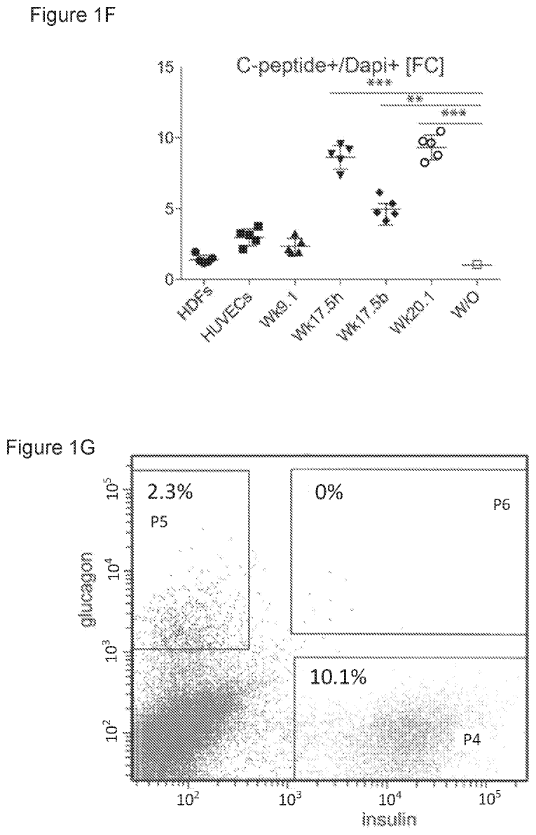

Human EPs are first detected during development between weeks 7 and 8 (Wk7-8) by expression of the pro-endocrine gene NGN3. EPs expand around Wk12-13 when islets become vascularized, and mature around Wk26-29 (Piper, 2004). The inventors therefore obtained human pancreas and other endodermal organs at Wk9.1, 10.6, 13, 14.6, 16.3 (separated as body and head of pancreas, 16.3b and 16.3h), 17.5 (17.5b and 17.5h), and 20.1, and isolated M-E cells to derive 9 stage- and organ-specific human primary cell lines (FIG. 1A and Experimental Procedures).

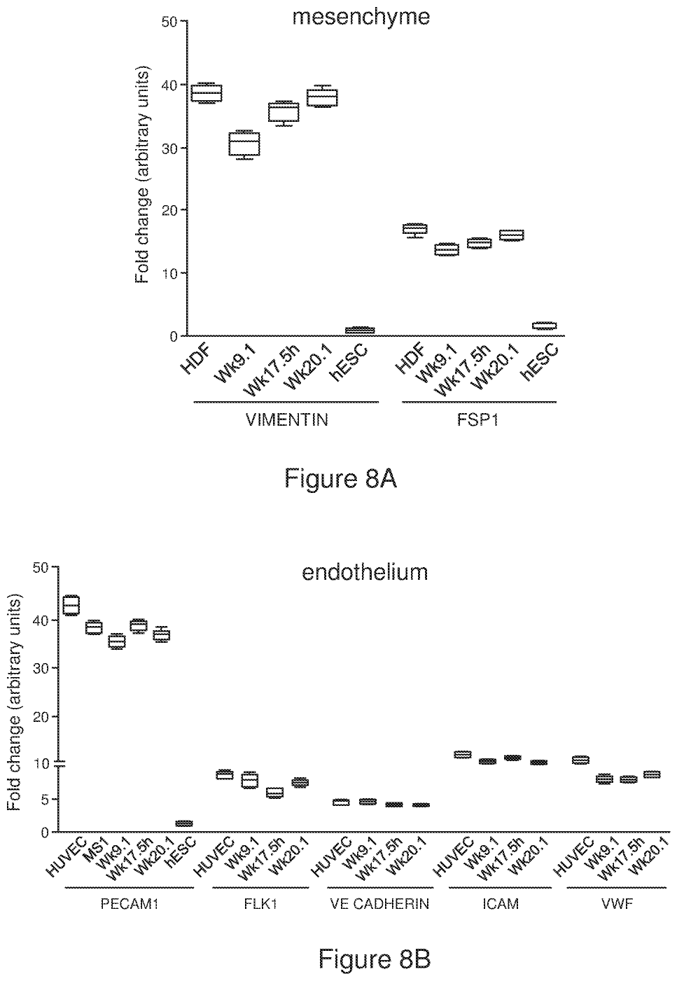

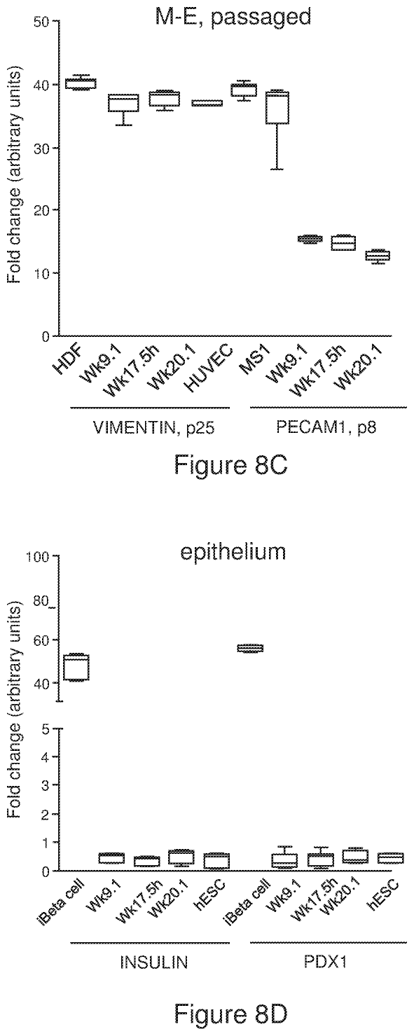

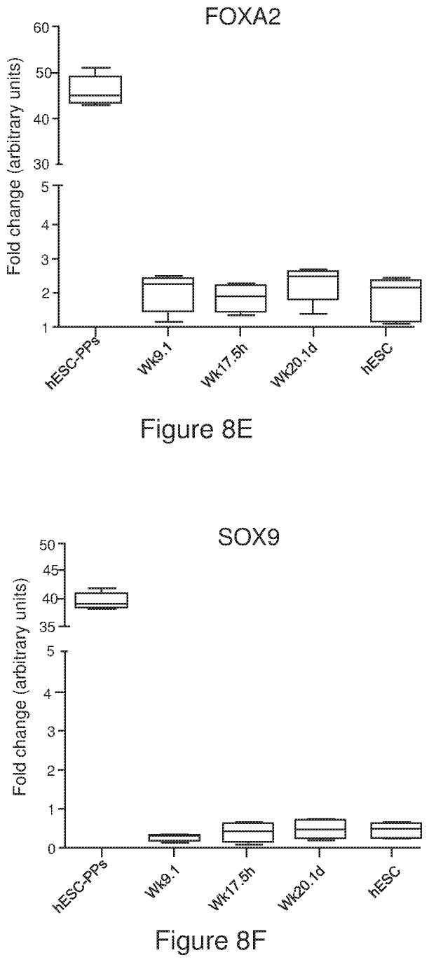

To characterize the de novo established M-E cell primary lines, immunofluorescence was used to assess expression of mesenchymal (Vimentin) and endothelial markers (PECAM1, CFIII) (FIG. 1B). qPCR was also used to quantify mesenchymal markers (Vimentin, FSP1) (Franke et al., 1982; Franke et al., 1978; Strutz et al., 1995) and endothelial markers (PECAM1, FLK1, VE Cadherin, ICAM, and VWF) (Albelda et al., 1991; Breier et al., 1996; Durieu-Trautmann et al., 1994; Jones et al., 1981; Lawson and Wolf, 2009; Yamaguchi et al., 1993) in the derived primary cells. The inventors then compared the observed expression levels to those of mesenchymal character (human dermal fibroblasts, HDFs) and endothelial character (human umbilical vein endothelial cells, HUVECs (Jaffe et al., 1973) and murine pancreatic endothelial cells, Mile Sven1, MS1 line (Arbiser et al., 2000)(FIG. 8A-8B). There was enrichment of all analyzed markers in each cell line, illustrating the lines are specifically of M-E origin. The expression of mesenchymal markers was maintained for at least 16 passages, but expression of endothelial genes decreased after 8 passages (FIG. 8C). Experiments were performed with primary M-E cells with early 6 passages. There was no expression of epithelial pancreatic progenitor markers PDX1, SOX9, and FOXA2 (Ben-Shushan et al., 2001; Deutsch et al., 2001; Lioubinski et al., 2003; Ohlsson et al., 1993; Piper et al., 2002; Stahlman et al., 1998) or β cell marker INS in any of the established cell lines (FIGS. 8D-8F).

To determine whether the niche influences endocrine cell formation and differentiation, particularly in promoting progenitors to differentiate into β cells, and at which developmental stage, each of the M-E cells were co-cultured with hESC-derived PPs. The hESCs were guided in a stepwise manner towards pancreatic fate as outlined in FIG. 1C (Sneddon et al., 2012). The efficiency of differentiation was assessed at each step and the expression of FOXA2 and SOX17 for DE and PDX1 and NKX6.1 for PPs (FIG. 1C) was quantified. The hESC-derived PPs were co-cultured with 9 niche primary cell lines for 3, 7, or 14 days (FIG. 1D). To determine the specificity of the niche, PPs were co-cultured with HUVECs, HDFs, and mouse embryonic fibroblasts (MEFs), none of which are present in the developing pancreas in vivo. Co-culturing PPs with Wk17.5h and 20.1 niche for 7 days increased C-peptide+ cells (8- and 9-fold compared to PPs without co-culture (W/O) or in co-culture with HDFs), whereas co-culturing PPs with Wk9.1 niche increased C-peptide+ cells only 1-fold over W/O condition (FIGS. 1E-1F), as measured by quantifying immunofluorescent images (FIG. 1F). There was no further significant increase in the number of C-peptide+ cells after increasing the time of co-culture to 14 days. Similarity, there was no significant C-peptide+ cell induction after 3 days of co-culture. 1 cells were then evaluated after co-culture with most efficient pancreatic niche cells, namely Wk17.5h and Wk20.1.

Two criteria were used to determine the maturity of hESC-derived β cells after 7-day co-culture with Wk17.5 and Wk20.1: flow cytometry to determine the amount of polyhormonal or non-INS+ cells, and GSIS to determine their functionality. The inventors obtained 10.1% INS+ cells, 2.3% glucagon (GCG)+ cells, and importantly no cells co-expressing both hormones (FIG. 1G). Thus, progenitors cultured with the pancreatic niche differentiate primarily into 1 cells, with no immature, polyhormonal cells persisting.

### Example 2

### Human B Cells Induced by Pancreatic Niche are Glucose Responsive in Vitro

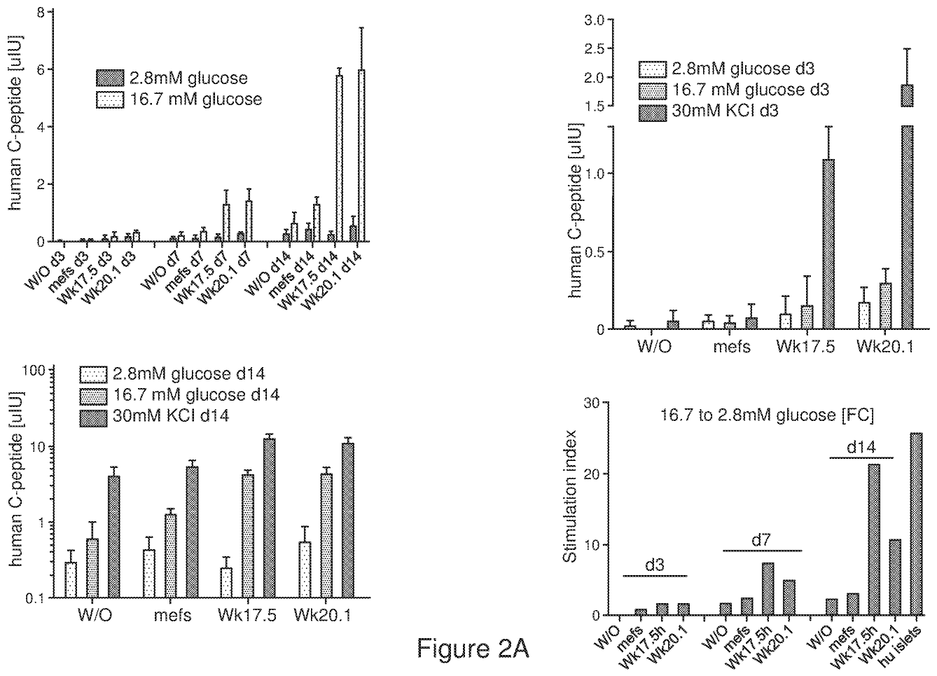

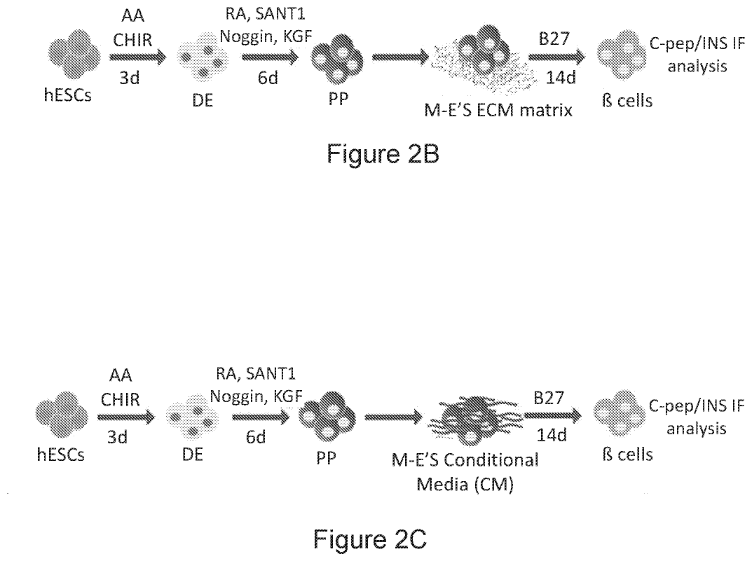

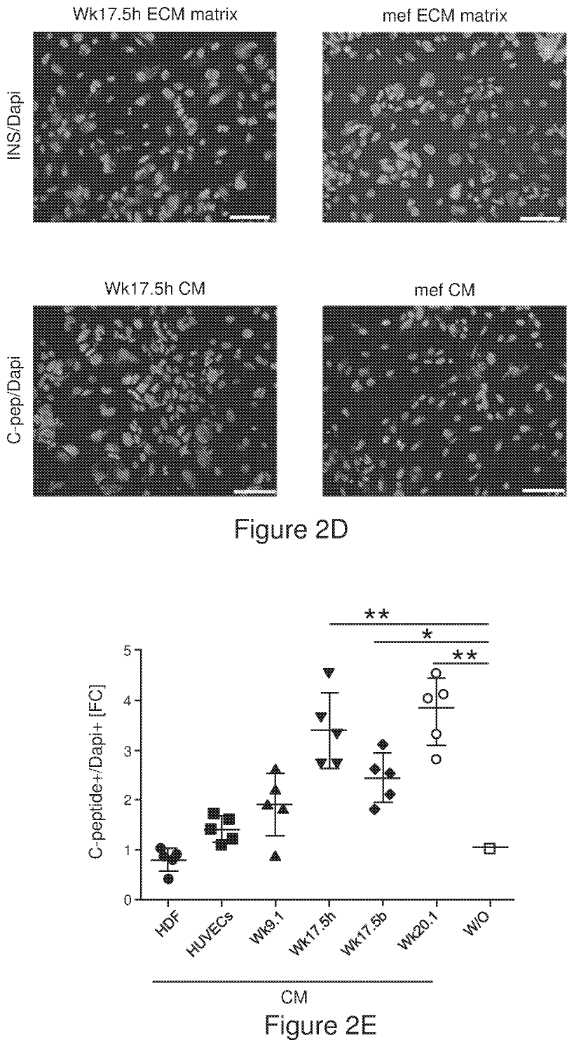

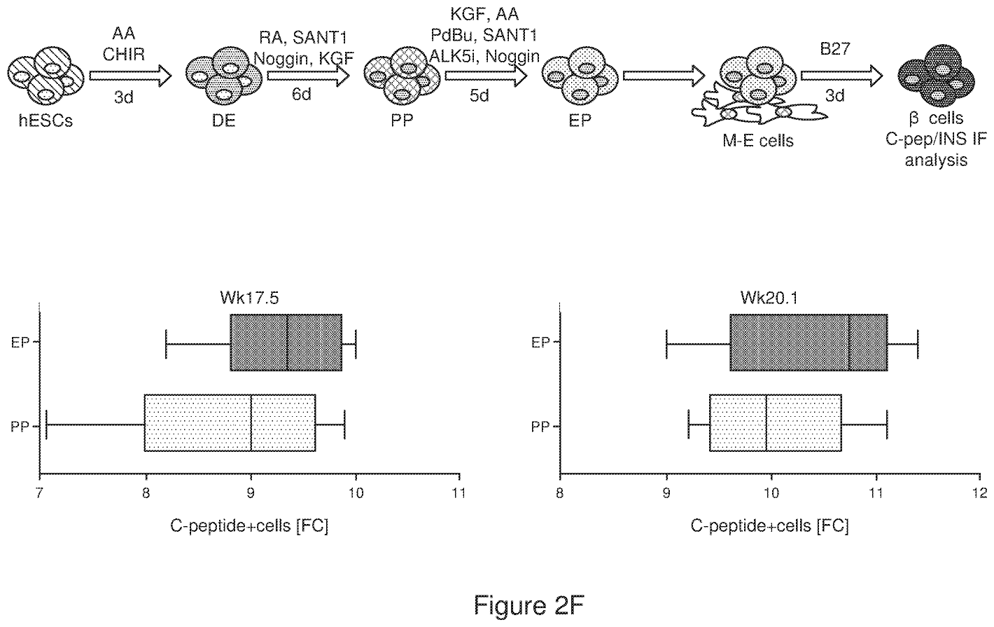

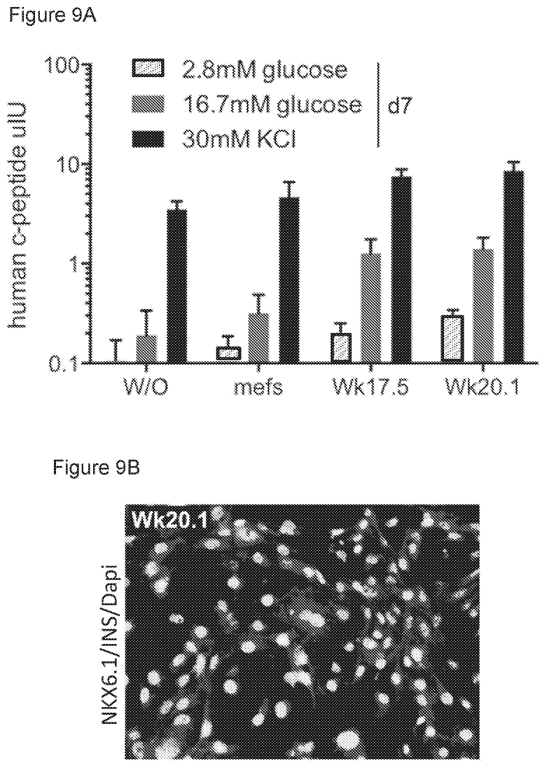

In vitro-derived β cells do not yet show comparable to native cells glucose-induced INS secretion (GSIS), which is the key function of the cells lost in diabetes. To assess the physiological capacity of these cells, GSIS was performed after coculturing progenitors with the Wk17.5 and Wk20.1 niche for 3, 7, and 14 days and the amount was determined of secreted human C-peptide, a byproduct of insulin production. The GSIS is designed to mimic basal blood glucose levels (2.8 mM), a level at which naïve β cells are not stimulated, and elevated blood glucose levels (16.7 mM) to stimulate β cells to secrete C-peptide. After 7 and 14 days of co-culture, but not as early as 3 days, cells gradually responded to stimulation with a high concentration of glucose (FIG. 2A). However, at day 7 the cells were still not able to release C-peptide specifically in response to elevated glucose levels (16.7 mM) (FIG. 9A). The greatest response as measured by secretion of C-peptide was detected at day 14 in all tested conditions, but day 14 co-culture with Wk17.5h niche had the highest C-peptide release to high glucose levels (FIG. 2A″). The distinct outcomes from each co-culture support the embodiment that the niche temporally regulates endocrine cell differentiation.

To better evaluate the maturity of P cells differentiated in coculture with niche cells, β cells were stimulated with 30 mM KCl, which hyperpolarizes the cell membrane, allowing measurement of stored c-peptide. Co-culturing PPs with selected M-E cells for 3 days is insufficient to form mature β cells, though the cells did produce a small amount of C-peptide after KCl stimulation (1.2 μIU/103 C-peptide/cell for Wk17.5 and 1.8 μIU/103 C-peptide/cell for Wk20.1) (FIG. 2A′). More C-peptide was detected at days 7 and 14 of co-culture after stimulation with KCl, 6 μIU/103 and 13 μIU/103 C-peptide/cell for day 7 and 14, respectively (FIGS. 2A″ and 9A).

Cells co-cultured with stage-specific M-E gained the ability to sense glucose and respond by secreting the appropriate amount of C-peptide by day 14. At that day, only β cells derived in co-culture with Wk17.5h and 20.1 niche responded adequately to fluctuating glucose levels. Cells cultured with Wk17.5h niche had the greatest sensitivity to low and high glucose levels, with a 20-fold difference compared to no-coculture or 10-fold to mefs-coculture controls (FIG. 2A′″). The inventors also performed identical GSIS tests on four independent batches of human cadaveric islets and detected a stimulation index, defined as ratio high-to-low-glucose induced C-peptide secretion, ranging from 17-29 (FIG. 2A′″). Together, these data show that 0 cells derived in presence of primary pancreatic stage specific, M-E cells are functionally comparable to human cadaveric islets. Finally it was confirmed that INS+ cells induced in presence of stage specific primary M-E cells co-express other β cell marker NKX6.1 (FIG. 9B), a signature of mature β cell.

### Example 3

### Paracrine Factors and ECM from Mesenchyme and Endothelial Cells Induce Human Endocrine Cell Differentiation In Vitro

Crosstalk between the pancreatic niche and progenitors can be achieved through secreted factors, cell-cell interactions, and ECM. Because the co-culture experiment permitted direct cell-cell contact, the inventors performed separate co-culture of PPs with ECM and secreted factors of M-E primary cells to avoid cell-cell contact and to dissect how M-E cells promote β cell differentiation. To evaluate the effect of ECM, M-E cells were cultured for one week and then removed from the plate, leaving only the secreted ECM on the dish (ECM matrix). PPs were later seeded on the same dish and co-cultured for 10 days (FIG. 2B). To determine the effect of soluble secreted factors alone, supernatants were collected from M-E cells (M-E conditional media/CM) and were applied to PPs for 14 days (FIG. 2C). Both the supernatant and ECM promoted β cell differentiation, which was detected by staining for C-peptide and INS (FIG. 2D). Although there was enhanced β cell differentiation from ECM and secreted factors, the increase in C-peptide+ was lower than in co-culture with primary cells (FIGS. 1F and 2E).

In the weeklong co-culture of PPs with M-E primary cells, the PPs must traverse through the EP stage to become β cells. Therefore, it was next tested when the Wk17.5 and 20.1 M-E cells had greatest impact on β cell differentiation by co-culturing PPs or EPs with M-E cells for 7 days or 3 days, respectively. Co-culturing EPs with Wk17.5 or 20.1 M-E primary cells for 3-days leads to a 7-8-fold increase in β cell induction, as measured by increase in C-peptide+ cells and compared to EPs differentiated without co-culture (FIG. 2F), thus indicating that these M-E primary cells efficiently induced 1 cells from EPs and PPs and indicating that M-E cells may exert their primary effect on EPs.

In summary, the molecular environment within the human pancreatic niche varies at each stage, with niche cells from specific developmental stages promoting endocrine maturation through the secretion of soluble factors and ECM.

### Example 4

### Identification of Secreted Growth Factors and ECM Components from M-E Cells

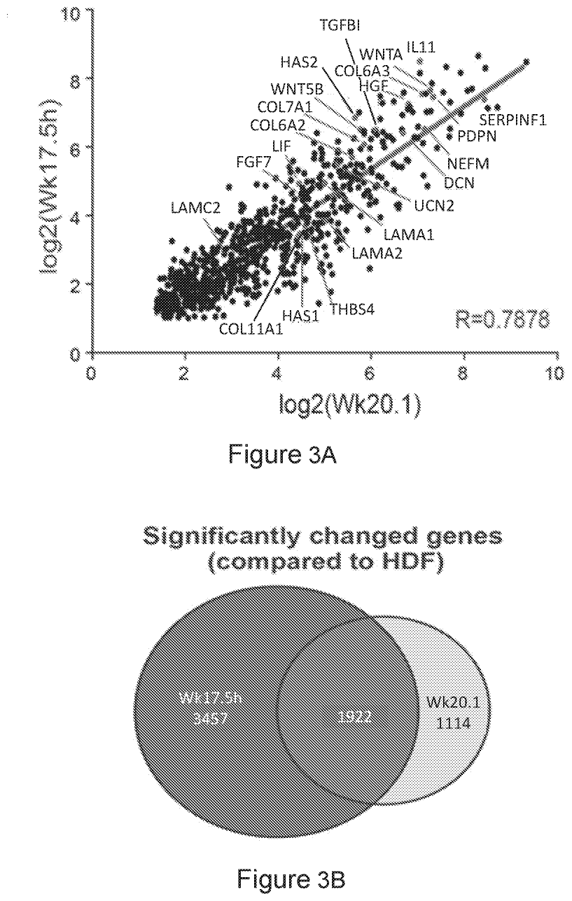

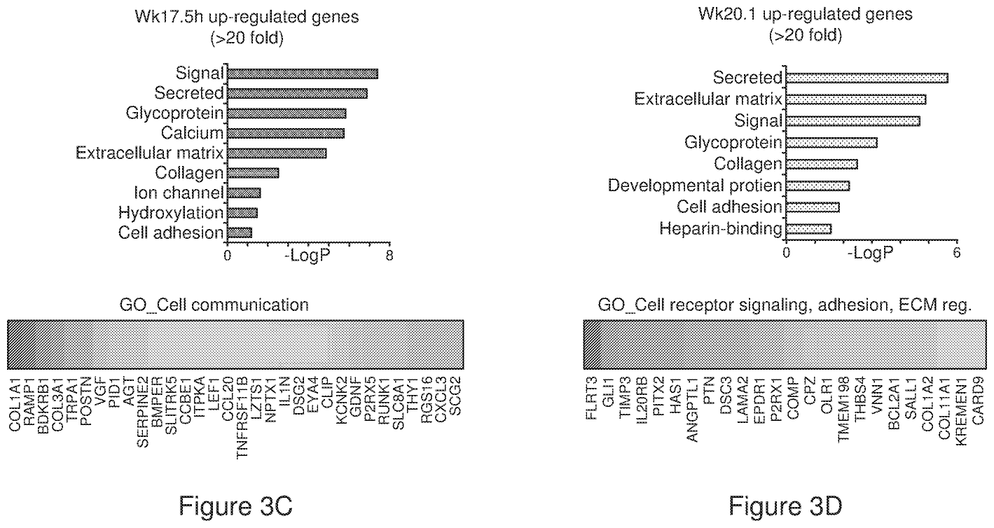

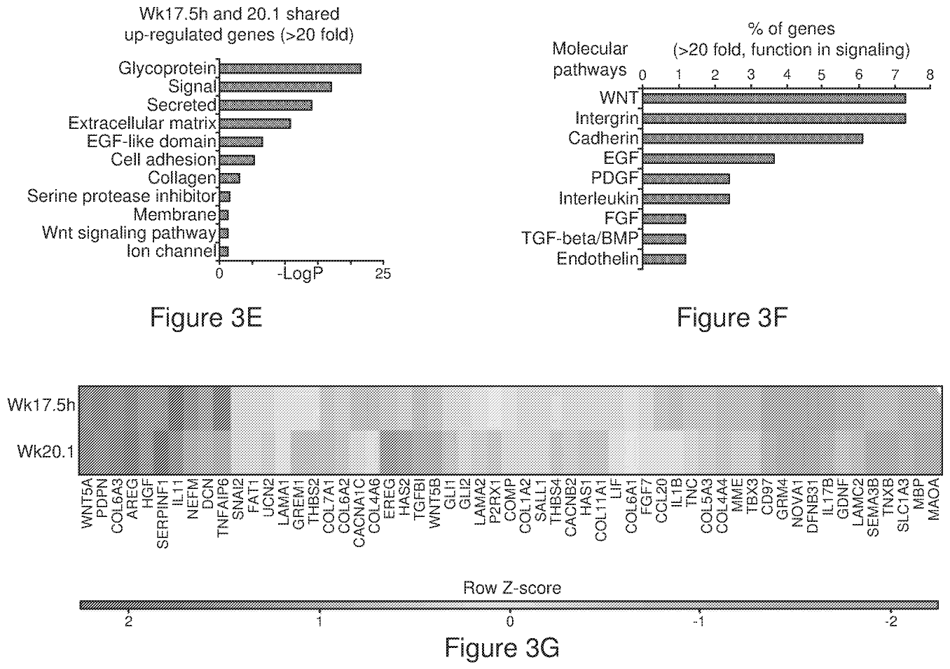

A gene expression array was conducted to identify candidate factors enriched in these pancreatic niche cells compared to HDFs. Pearson's correlation (R²=0.7878) was used to illustrate the degree of linear dependence between Wk17.5h and 20.1 cell gene expression profiles, and the similarity was confirmed between these two times points, yet some distinctions in gene expression profiles did exist (FIG. 3A). Compared to HDFs, there were 3457 genes significantly changed in Wk17.5h and 1114 in Wk20.1 cells. It was also found that there are 1922 genes that are significantly changed in both primary cell lines as compared to control HDFs (FIG. 3B), although there was variability in their expression levels. Functional annotation of upregulated genes enriched in Wk17.5h or Wk20.1 showed that many of the genes coded secreted proteins or ECM components and were involved in cell signaling (FIGS. 3C-3D). Importantly, a number of enriched secreted factors and ECM components were conserved between both Wk17.5 and Wk20.1 cells that may play a role in the crosstalk between the niche and the epithelium (FIG. 3E). Within the signaling gene ontology (GO) category, the most enriched pathways in both cell types were the WNT, Integrin, and Cadherin pathways (FIG. 3F). The inventors compared the Wk17.5h and 20.1 expression profiles of transcripts known to function as secreted factors or ECM components (FIG. 3G). Although there were a few genes that showed different expression levels, such as GLI1, GLI2, GREM1 and THBS2, many genes showed the same expression trend (FIG. 3G). ECM-related genes including COL7A1, COL6A3, LAMA1, LAMA2, HAS1, and HAS2, and secreted factors such as WNT5A, FGF7, SERPINF1, PDPN, HGF, LIF, Endocan, UCN2, and DCN were significantly upregulated in both Wk17.5h and 20.1 compared to HDFs (FIG. 3G). Some genes, such as LAMA1 and LAMA2, were previously shown to improve β cell differentiation in vitro (Jiang et al., 1999; Russ et al., 2016). Many of the factors, however, have heretofore unknown roles in pancreatic development and the inventors set out to investigate their contribution to human endocrine differentiation.

### Example 5

### Growth Factors in the Human Pancreatic Niche Differentiate EPS into INS-Secreting Cells

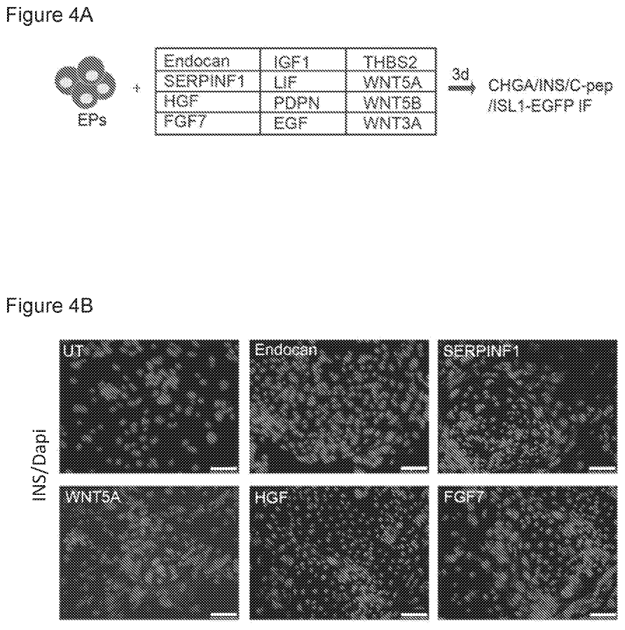

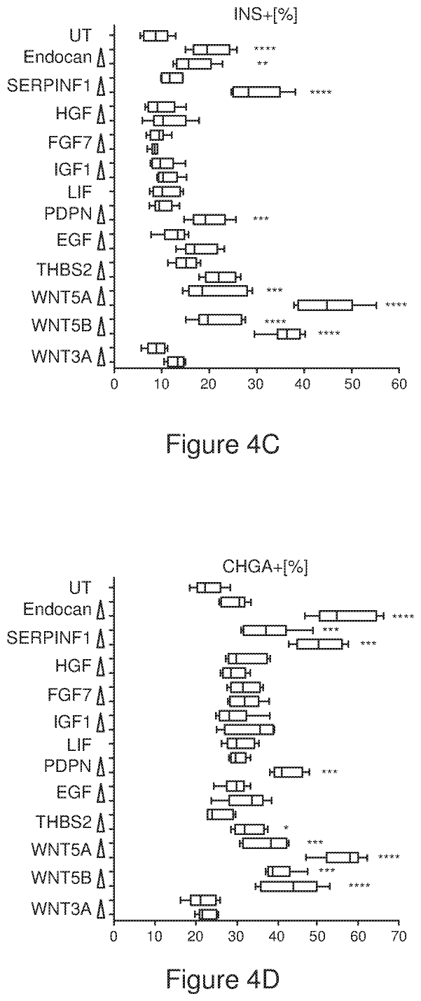

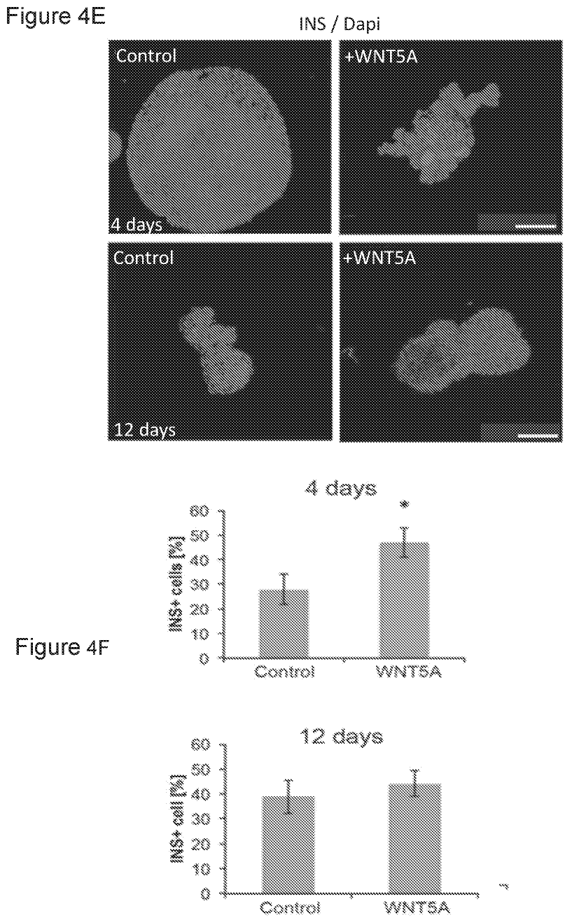

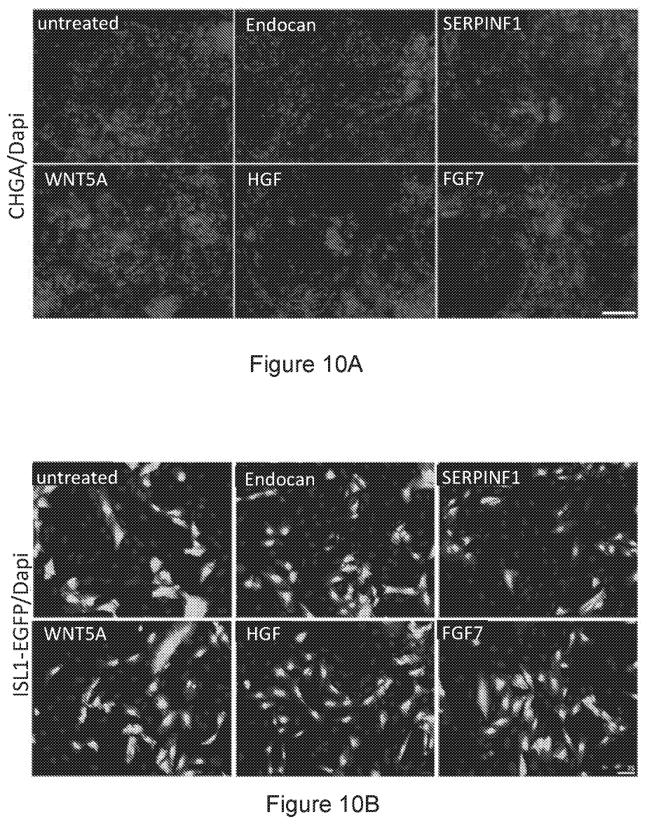

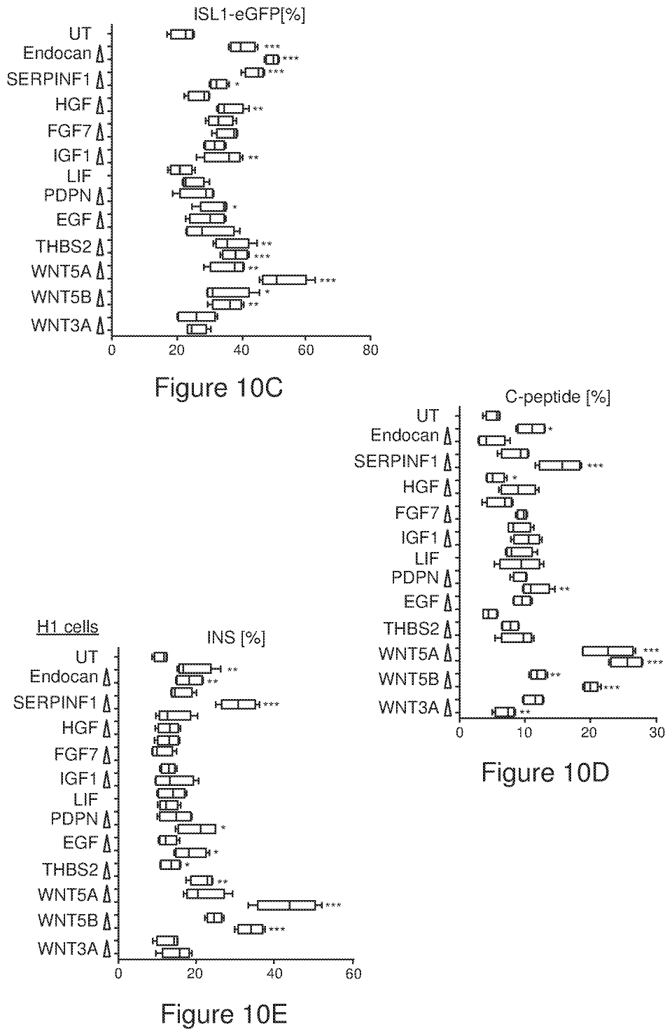

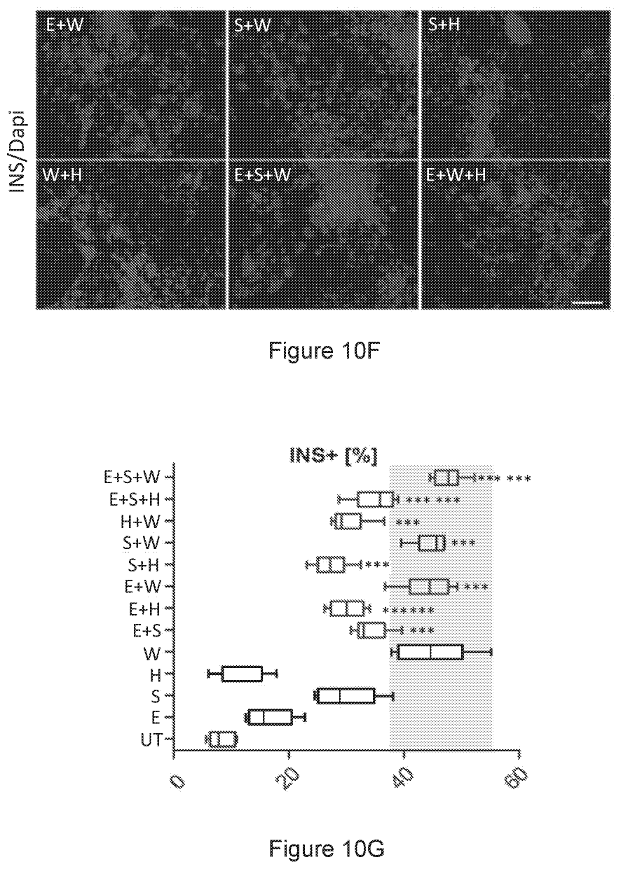

Based on the microarray analysis, the inventors selected factors upregulated in both Wk17.5h and 20.1 M-E cells, including FGF7, HGF, PDPN, SERPINF1, WNT5A, THBS2, IGF, Endocan, LIF, and WNT3A, to test their role further in human β cell differentiation in vitro. As the co-culture with M-E had a positive effect on EP differentiation and the signals responsible for endocrine cell maturation are not well understood, hESC-derived EPs were used to study the role of niche derived-secreted factors in differentiating these cells into mature β cells (FIGS. 2F and 4A). The inventors aimed to identify factors that increase EPs by measuring the expression of EP markers chromogranin A (CHGA) and Islet-1 (ISL1) (FIGS. 4A-4D and 10A-10E), and factors that enhance β cell specification by testing the expression of β cell markers, INS and C-peptide (FIGS. 4B-4C and 10).

hESC-derived EPs were treated separately with selected factors at two different concentrations for 3 days before assessing CHGA, ISL1, INS and C-peptide expression by immunofluorescence (FIGS. 4A-4D, 10, Table 3). To gain further insight into differentiation efficiency, a human ISL1^{Cre/+}; pCAG^{loxP-STOP-loxP-EGFP} hESC line engineered to monitor ISL1 through EGFP and to permit lineage tracing of ISL1+ cells was utilized (FIG. S3A) (Bu et al., 2009). Some factors, like HGF, IGF, and THBS2 increased the number of EPs by increasing the number of ISL1+ cells (˜2-fold after high concentration treatment) (FIG. 10B), but had little or no effect on the number of CHGA+ and INS+ cells (FIGS. 4C-4D and 10C), i.e. they showed an effect on EP expansion or induction but not differentiation.

Cells treated with Endocan, SERPINF1, WNT5A, and PDPN had increased numbers of ISL1-eGFP, CHGA+, INS+ and C-peptide-+ cells over untreated controls (FIGS. 4B-4D, 10C-10D), demonstrating not only an increase in expansion/induction, but also differentiation. WNT5A had the strongest effect and caused on average 5-fold increase in number of cells positive for all test markers. Treatment with WNT5B, a WNT5A paralog, also increased INS+ cell numbers in a dose-dependent manner (FIG. 4C), although the increase was not as marked as seen with WNT5A. PDPN, which encodes podoplanin, a mucin-type transmembrane glycoprotein whose function is not fully determined, increased the number of INS+ and CHGA by 3-fold at high concentration (FIGS. 4C-4D and 10A). Interestingly, Endocan caused significant increase in number of CHGA+ cells but had moderate effect on induction of INS+ cells (FIGS. 4C-4D and 10D-E). As CHGA is pan-endocrine marker, the PDPN may be beneficial for the specification of endocrine cells other than β cells, in specific embodiments. The effects of selected growth factors were confirmed using another hESC line, H1, which showed similarly increased CHGA+ and INS+ cell numbers (FIG. 10E). These experiments identified several known and novel growth factors secreted from the human pancreatic niche that substantially expand the EP pool or differentiate EPs into INS+ cells. WNT5A also facilitated in β cell differentiation by an independent protocol (Pagliuca et al., 2014), increasing the number of INS+ cells in just 4 days of treatment, compared to INS upregulation at 12-day in original protocol (FIG. 4E), indicating that WNT5A effectively promotes β cell formation regardless of the differentiation strategy.

### Example 6

### A Combination of Niche-Derived Growth Factors Improves Endocrine Specification and B Cell Induction in HESC-Derived EPS

As the pancreatic niche exhibits complex signal signatures in a temporally and spatially specific manner in vivo, it was investigated how these factors cooperate to promote differentiation of human endocrine cells. To assess the cooperation between factors in an efficient manner, four factors were selected that promoted endocrine differentiation (Endocan, SERPINF1, WNT5A and HGF) and their effect was tested on hESC-derived EPs in combinations of two or three and at various concentrations (Table 4). After 3 days of treatment, the number was evaluated of INS+ cells compared to untreated cells or treated with single growth factor (FIG. 10D). Combination of Endocan, SERPINF1 and/or WNT5A further enhanced the INS expression. Endocan and HGF have enhanced effects on INS+ cell number when combined with WNT5A and SERPINF1, however neither factor was more beneficial than WNT5A alone (FIG. 10G). Interestingly, of all the factors and combinations tested, treatment with WNT5A in different combinations at a concentration of 500 ng/ml consistently and most significantly increased the number of INS+ cells (FIG. 10D) and therefore the inventors set out to study the role of WNT5A in endocrine differentiation into β cells.

### Example 7

### WNT5A is Present in the Human Pancreatic Niche and Sufficient to Induce B Cell Markers in EPS

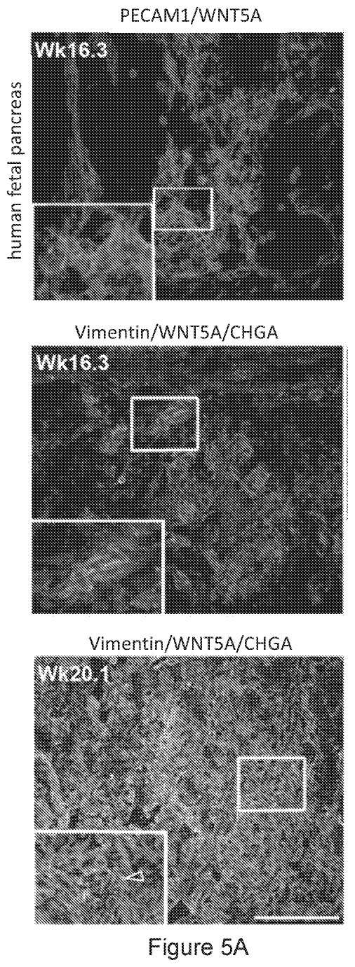

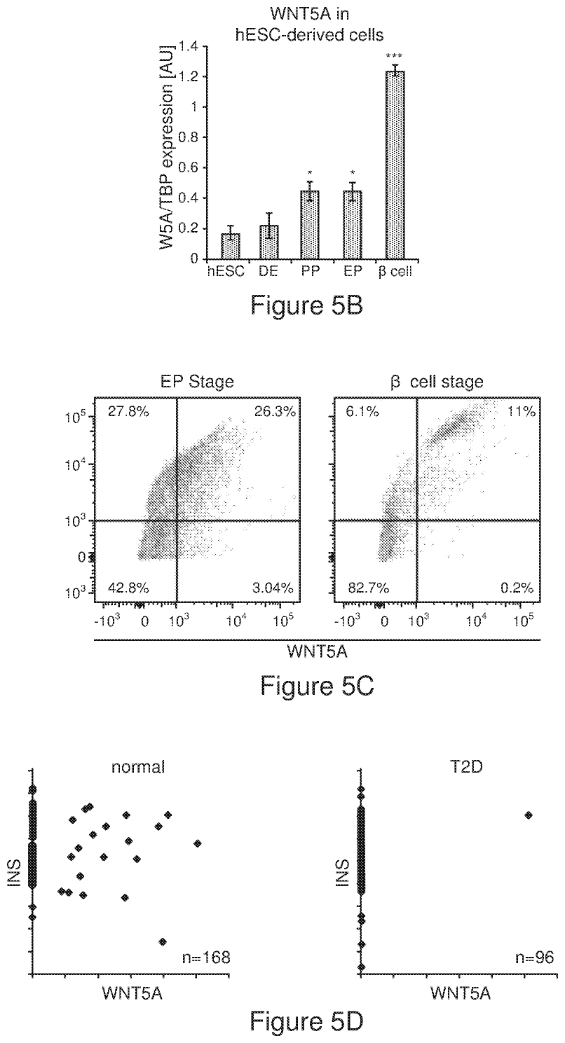

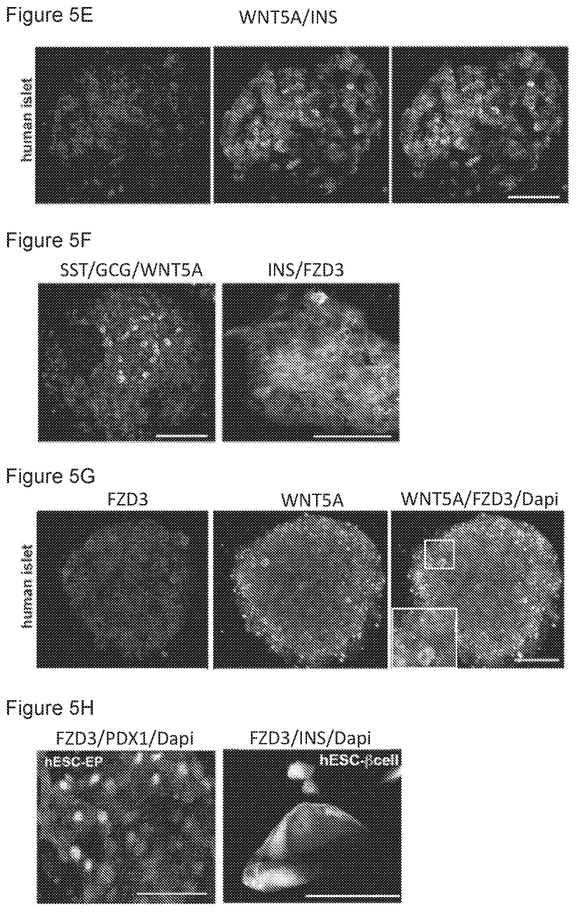

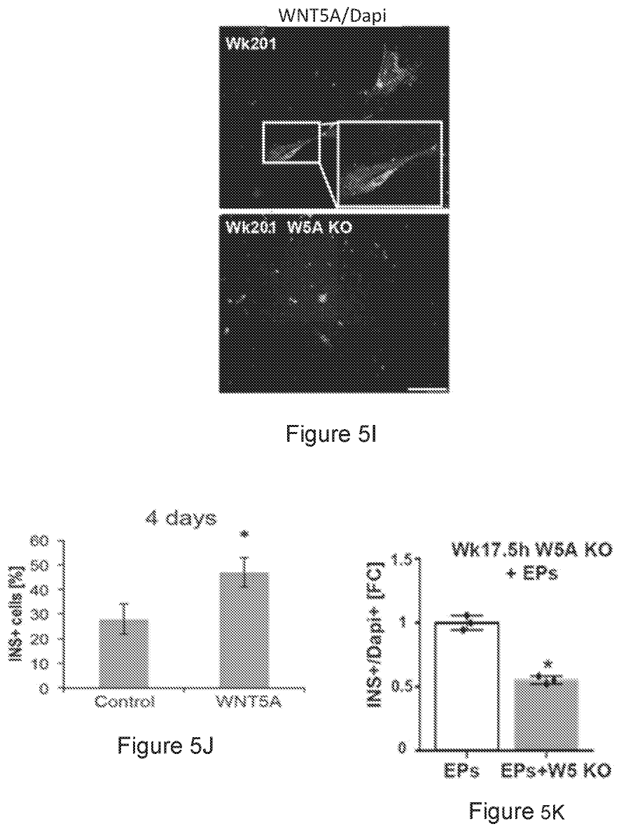

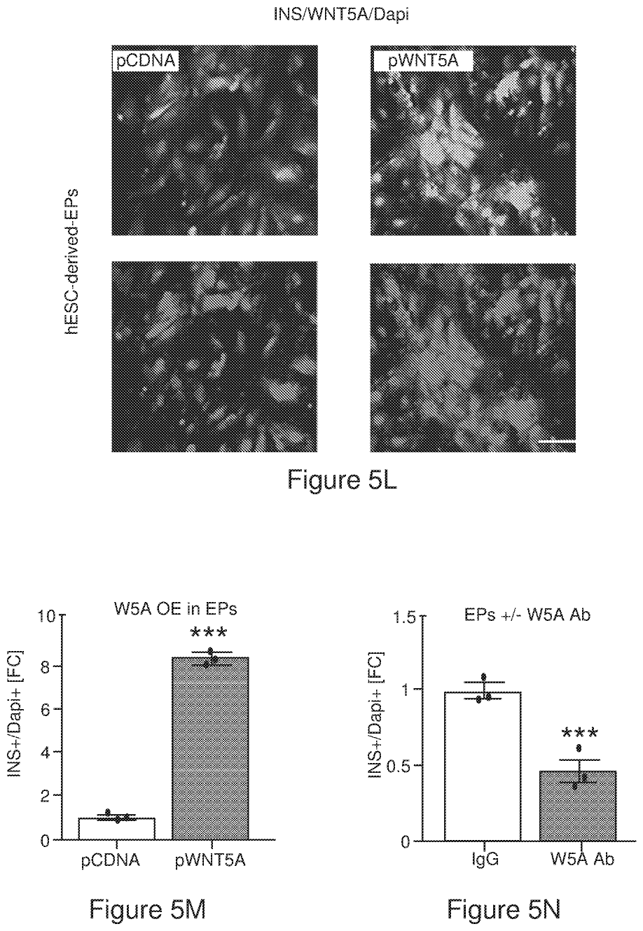

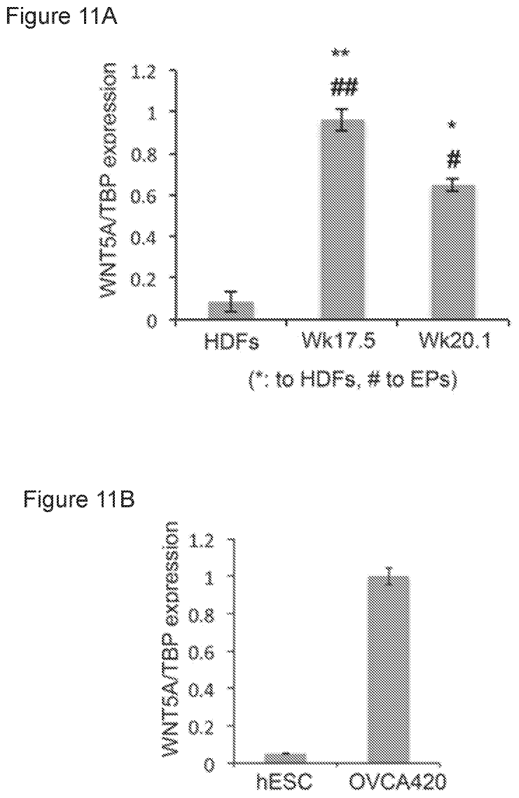

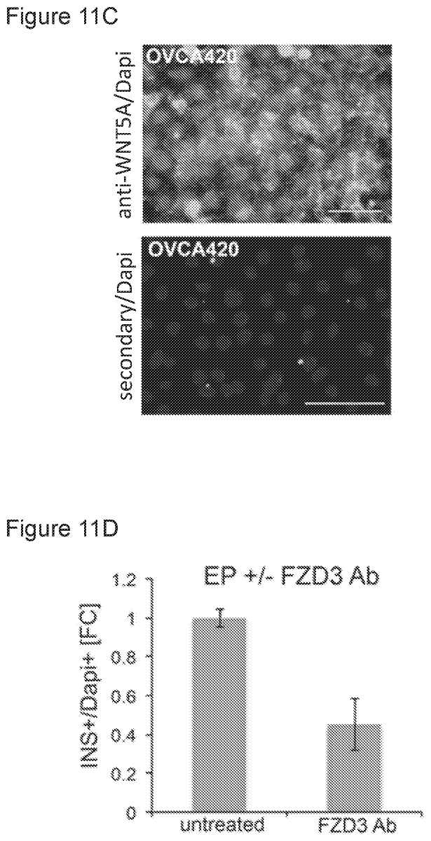

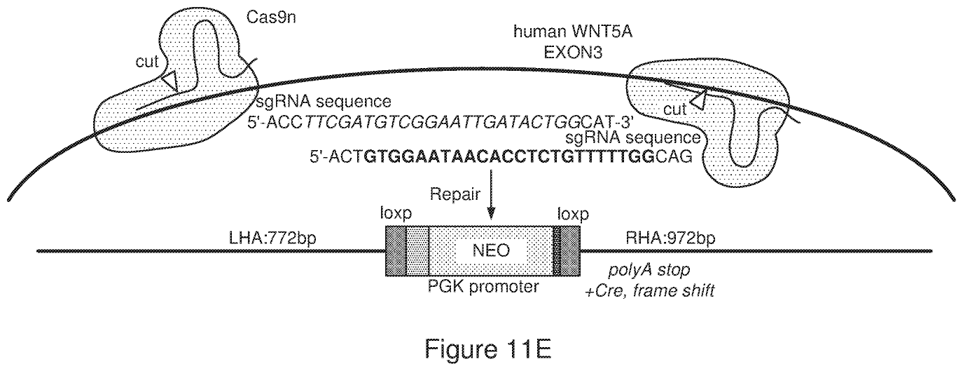

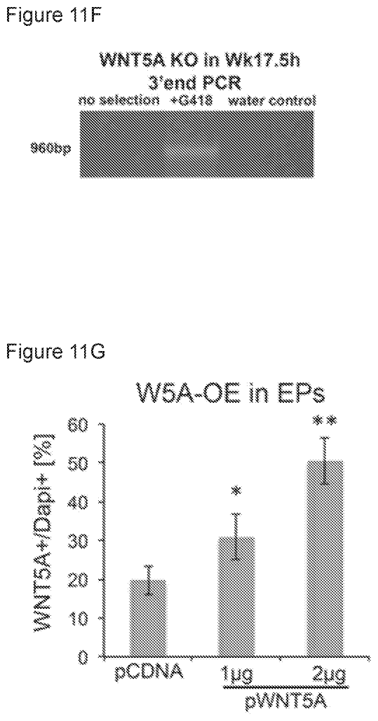

WNT5A is expressed in the mouse pancreatic mesenchyme at e11.5 and is thought to play a role in islet formation (Heller et al., 2003; Kim et al., 2005). Recent studies showed that WNT5A induces proliferation of some β cells and β cell maturation (Bader et al., 2016), but the role of WNT5A in EP differentiation is not well understood. Immunostaining was performed for WNT5A and other pancreatic markers on human fetal pancreatic tissue from Wk16.3 to 20.1, as well as qPCR with different M-E primary cells (FIG. 11A). The inventors identified WNT5A expression in the pancreatic niche specifically in Vimentin and PECAM-1 positive M-E cells at Wk16.3, however at Wk20.1 WNT5A was expressed primarily in developing endocrine cells as marked by CHGA expression (FIG. 5A). The specificity of WNT5A antibodies was confirmed by staining human OVCA420, ovarian cancer cell line, that highly expresses WNT5A (Ford et al., 2014) (FIGS. 11B-11C) The dynamic expression of WNT5A, with higher expression in the pancreatic niche at an earlier stage, and then later in EPs, correlates with the positive influence of paracrine signaling from the M-E co-culture on EP differentiation until WNT5A expression becomes autocrine in committed endocrine cells at Wk20.1.

To characterize WNT5A expression throughout different stages of human endocrine development in vitro, its expression was examined in hESCs and hESC-derived DE, PPs, EPs and β cells using qPCR (FIG. 5B) and FACS analysis (FIG. 5C). WNT5A transcript expression increased in the EPs and β cells compared to hESCs, but not in DE or PPs (FIG. 5B). Consistent with these transcriptional changes, 50% of endocrine CHGA+co-expressed WNTSA and later on, 70% of INS+ cells coexpressed WNTSA (FIG. 5C). WNTSA is therefore present in the pancreatic niche, but later shifts to endocrine cells in vivo and in vitro. Interestingly, WNTSA was shown recently to be expressed in a subpopulation of mouse β cells and to stimulate β cell proliferation, the inventors therefore analyzed transcriptomes of single islet cells from healthy and type 2 diabetes (T2D) patients for WNT5A expression (Lawlor et al., 2017) (FIG. 5D). This analysis demonstrated that WNT5A is expressed in subpopulation of human β cells and its expression is almost completely lost in T2D patients. It was further investigated whether WNTSA was expressed in human adult islets as its expression may indicate it plays a role in 3 cell identity and/or function and it was found that WNT5A was co-expressed with most, but not all INS+ cells (FIG. 5E), supporting the proposed mouse model of β cell heterogeneity (Bader et al., 2016). Co-staining of GCG or somatostatin (SST) with WNT5A in human islets showed that WNT5A is very rarely expressed in oc nor 6 cells (FIG. 5F). The inventors then tested whether human endocrine cells are competent to respond to WNT5A signaling and detected expression of one of the potential WNT5A receptors, FZD3, in half of human β cells in the cultured islets (FIG. 5F′). Some β cells expressed WNT5A while the neighboring β cells expressed the FZD3 receptor, indicating possible signaling between adjacent β cells within islets (FIG. 5G). There was expression of FZD3 in hESC-derived PDX1+PPs and INS+β cells (FIGS. 5H-H′). To evaluate whether FZD3 is the main receptor responsible for transmitting the WNT5A signal, the inventors treated hESC-derived EPs with WNT5A and FZD3 neutralizing antibodies and observed a 2.5-fold decrease in the number of INS+ cells compared to EPs treated with only WNT5A (FIG. 11D). Together, PP and β cells expressed FZD3 and are therefore competent to respond to WNT5A secreted early from M-E cells and later from endocrine β cells.

To test the necessity and sufficiency of WNT5A in promoting human β cell differentiation, the inventors disrupted WNT5A expression in Wk17.5h and 20.1 cells by targeting the first constitutive exon (exon 3) using CRISPR-Cas9 nickase (WNT5A-KO). A neomycin cassette was inserted at exon 3 to introduce the frame-shift and to select for positive clones before confirmation of the knockout by external and internal PCRs (FIGS. 11E-11F) (Yang et al., 2016). The inventors confirmed the loss of WNT5A by immunofluorescent staining (FIG. 5I). Co-culture of Wk17.5h WNT5A-KO cells with EPs decreased the INS+ cells 3.6-fold compared to control Wk17.5h cells (FIG. 5J). In order to create a paracrine source of WNT5A, the inventors ectopically expressed WNT5A in HDFs before coculture with EPs (see Extended Experimental Procedures). Ectopic overexpression of WNT5A doubled the number of INS+ cells in EPs compared to controls cocultured with HDFs overexpressing the backbone plasmid (FIG. 5K).

To determine the sufficiency of WNT5A to influence the development of β cells, the inventors overexpressed and repressed WNT5A signaling in hESC-derived EPs. Overexpression of WNT5A in a dose-dependent manner was transiently introduced by 1 μg and 2 μg of pCDNA-WNT5A plasmid in EPs. Increased WNT5A+ cells were observed after 3 days of WNT5A overexpression, with verified dosage efficiency (FIG. 11G). The higher dose of pCDNA-WNT5A caused a 50% increase in WNT5A expression and 8-fold increase in the number of INS+ cells (FIGS. 5L-5M). Additionally, EPs were treated with 1 μg of WNT5A antibodies to block WNT5A signaling (Bilkovski et al., 2010) and β cell induction was evaluated. Blocking WNT5A signaling caused 2-fold decrease in number of INS+ cells, further indicating that WNT5A signaling has a significant impact on human β cell in vitro differentiation (FIG. 5N). Together, the bi-directional manipulations of WNT5A signaling suggested that secreted WNT5A in the pancreatic niche plays a crucial role in differentiating EPs into INS+ cells.

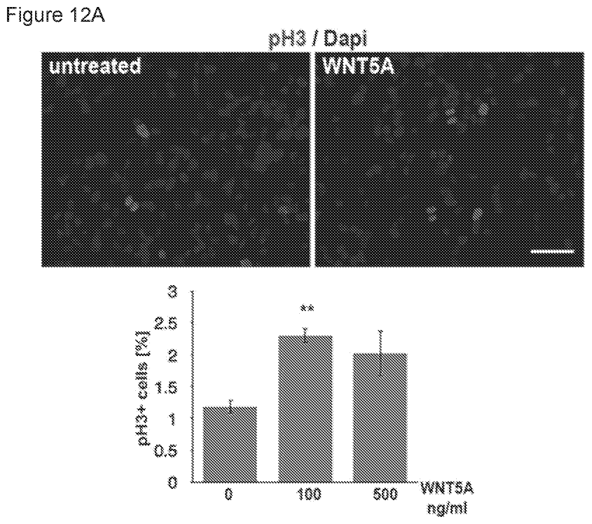

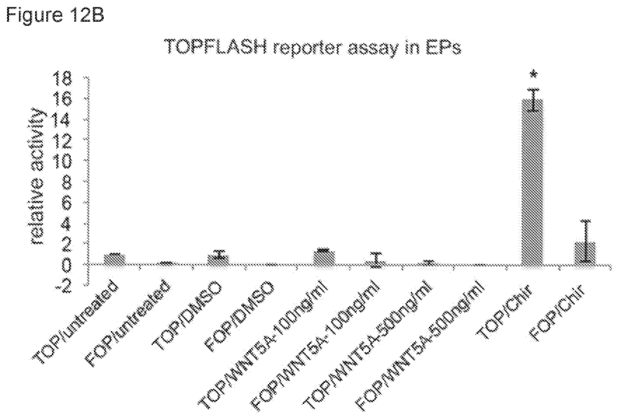

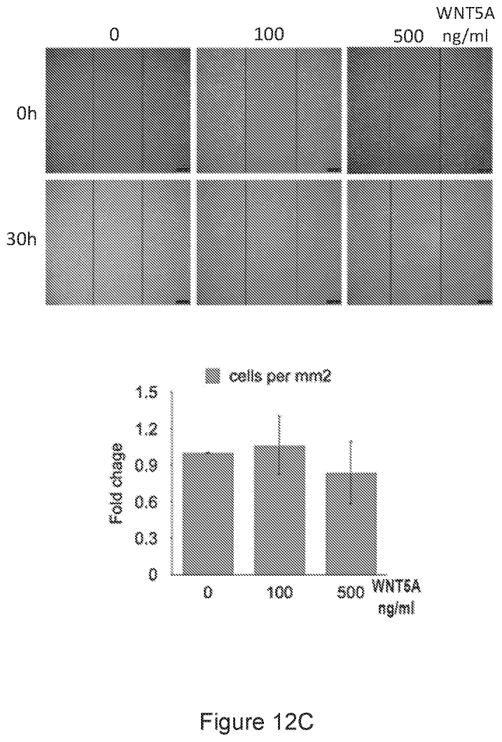

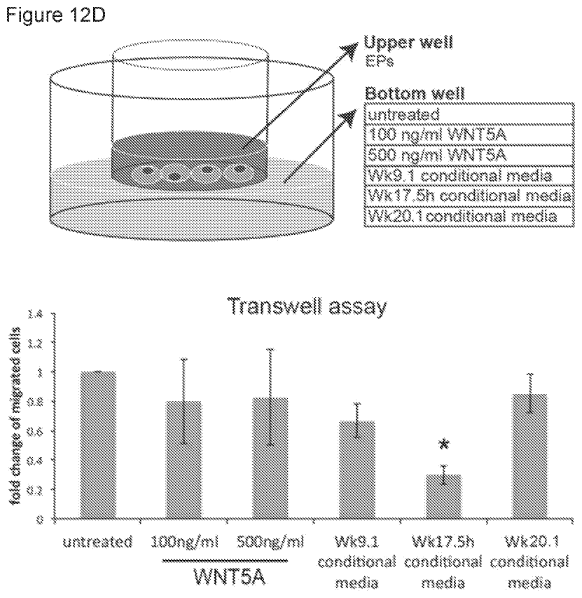

To determine whether WNT5A treatment increased INS+ cell numbers through proliferation or differentiation, the mitotic marker phospho-histone 3 (pH3) was utilized (FIG. 12A). Application of WNT5A increased pH3+ cells from 1% in control to 2%. When differentiation was tested after WNT5A treatment, the number of INS+ cells increased in a dose-dependent manner from 30% in B27 to 39% in 100 ng/ml of WNT5A and 60% in 500 ng/ml WNT5A, a much higher rate than that observed in proliferation. In specific embodiments, WNT5A increases INS+ cell numbers primarily through differentiation, with a minor effect on proliferation.

### Example 8

### WNT5A Acts Via Non-Canonical WNT and JNK Signaling During Pancreatic B Cell Differentiation

WNT5A activates the non-canonical and canonical WNT pathway (Mikels and Nusse, 2006; Torres, 1996). Here, it was first determined of the activity of the canonical β-catenin-dependent pathway in EPs after WNT5A treatment using the TOPFLASH reporter system (Veeman et al., 2003). EPs were transfected with either TOPFLASH or FOPFLASH and treated with WNT5A or GSK inhibitor CHIR99021 as a positive control. Untreated EPs had low TOPFLASH activity, and WNT5A treatment did not significantly activate or antagonize the 3-catenin-dependent pathway (FIG. 12B). Therefore, it was concluded that WNT5A is prone to utilize a non-canonical pathway in EPs, including the calcium and planar cell polarity (PCP)/JNK pathways (Kikuchi et al., 2012).

For islet formation, EPs must lose adherence and migrate from the epithelial layer (Kesavan et al., 2014; Kesavan et al., 2009). Because the non-canonical WNT pathway often affects cell motility and polarity downstream, it is possible that WNT5A affects EP migration and subsequently islet formation in specific embodiments, but there was observed no increase in cell mobility in scratch assays after WNT5A treatment (FIG. 12C). Based on the spatial expression of WNT5A in the surrounding mesenchyme and endothelium in vivo, it was considered that WNT5A could serve as chemoattractant for delaminating EPs. To test this consideration, a transwell assay was used to observe EP migration from the upper well with media toward the bottom well with WNT5A or Wk9.1, 17.5h or 20.1 M-E conditional media and it was found that WNT5A does not act as a chemoattractant in islet formation (FIG. 12D) (Boyden, 1962).

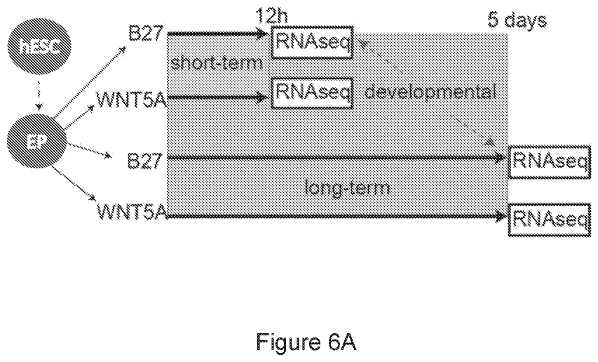

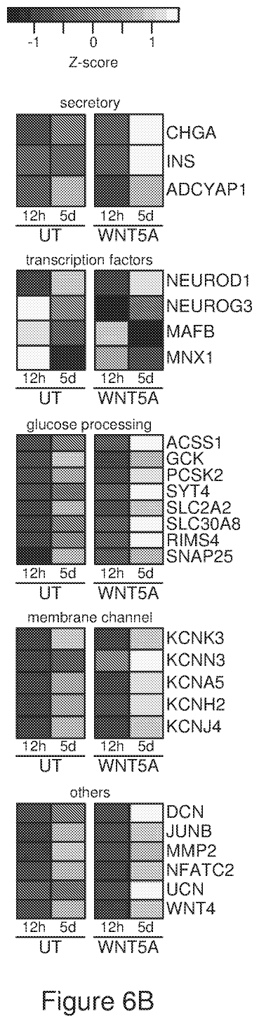

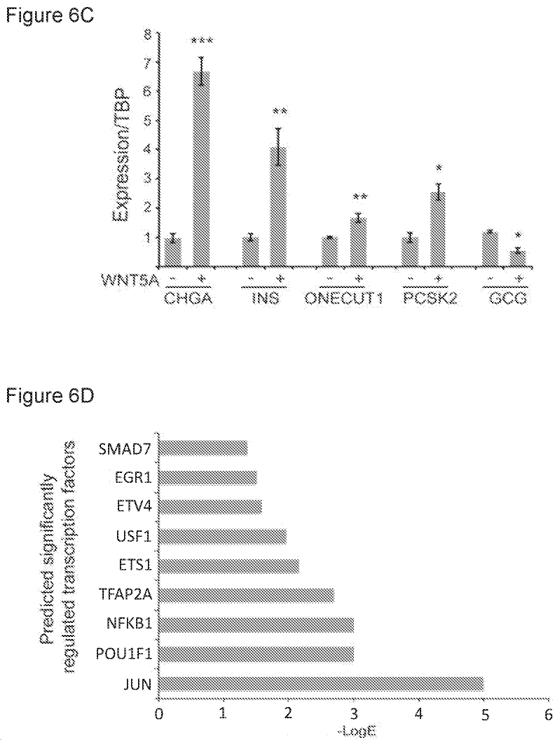

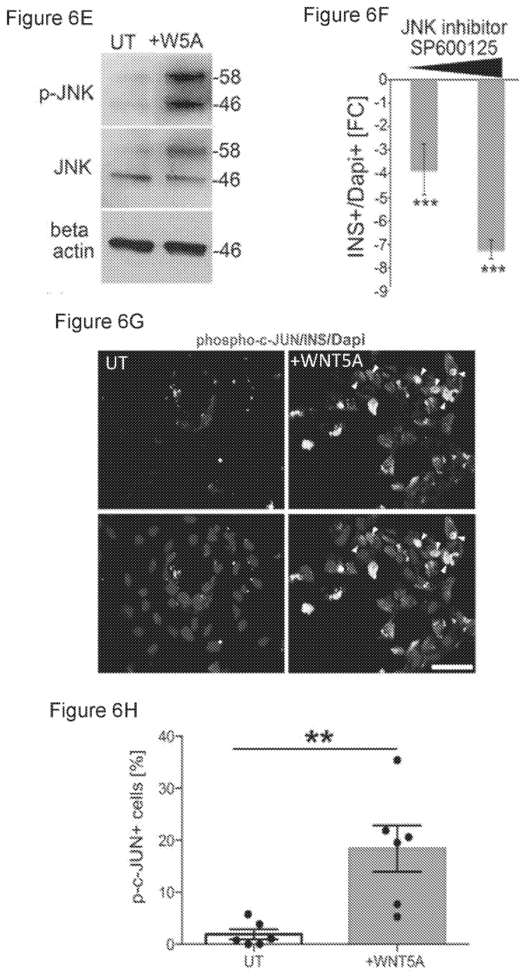

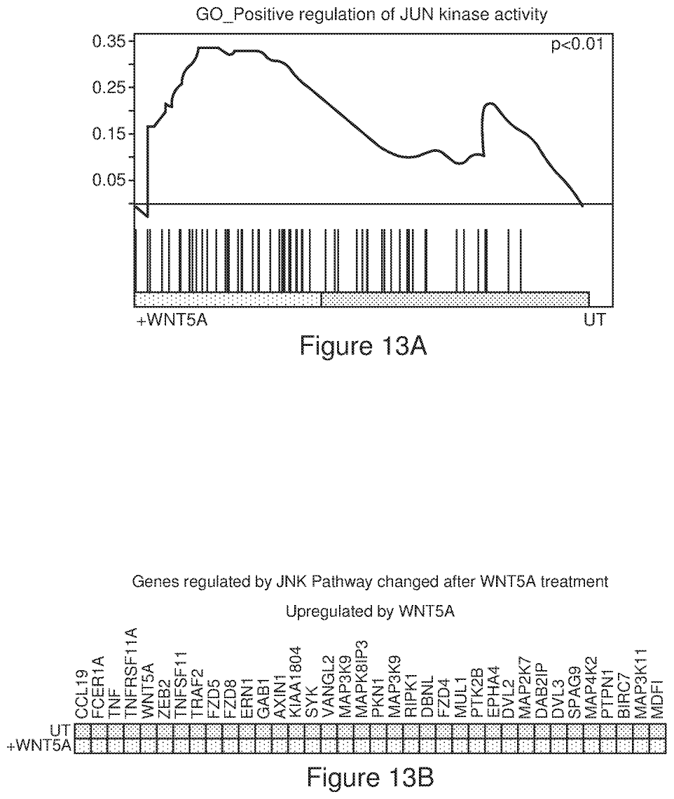

To investigate the downstream targets of WNT5A in EPs, RNA-sequencing was performed from cells treated with WNT5A over the short term (12h) and long term (5 days) (FIG. 6A). Sequencing of untreated (UT) cells and those treated with WNT5A for 12h (at the EP stage) and 5 days (at the β cell stage) uncovered developmental changes during in vitro differentiation. Of these changes, when 5 day-untreated cells were compared to 12h-untreated cells, up-regulation of progenitor and β cell markers including PDX1 by 3-fold, ONECUT2 and IAPP by 2-fold, and genes encoding membrane channels such as KCNN1 and SCN2A by 2-fold. These changes indicate that spontaneous β cell differentiation from EPs is limited in vitro. In contrast, 5-day WNT5A treatment caused the up-regulation of several β cell markers such as INS by 81-fold, transcription factors including NEUROD1 by 56-fold, glucose processing and insulin secretion regulators including GCK by 9-fold, PCSK2 by 7-fold and SYT4 by 16-fold (FIG. 6B), and downregulation ofMAFB by 3-fold, NGN3 by 2-fold and GCG-by 5-fold. Further verification of 5-day WNT5A-treated cells showed increased INS, CHGA, ONECUT1 and PCSK2, but decreased GCG expression compared to untreated cells (FIG. 6C). GSEA analysis determined a gene set associated with the JUN pathway to be significantly upregulated (FIG. 13A). Numerous genes shown to be regulated by JNK pathway were unregulated by WNT5A treatment (FIG. 13B) Pathway analysis was performed to predict regulation of known transcription factors based on upregulated and downregulated genes after 5 days of WNT5A treatment using TFactS, and it was found that JUN was the most significantly regulated transcription factor (FIG. 6D).

The inventors next looked at the JUN and PCP pathway as a putative downstream effector of WNT5A during β cell differentiation. Activity of JUN transcription factors is regulated by JNK-mediated phosphorylation. Short-term (12h) EPs treatment with WNT5A caused increased JNK expression and phosphorylation as determined by Western blot (FIG. 6E). The inhibition of JNK in EPs by small molecule SP600125 reduced INS+ cell number in a dose dependent manner, indicating that JNK plays an important role in β cell induction (FIG. 6F). The downstream JNK effector, c-JUN, was also hyper-phosphorylated (9-fold increase compared to untreated control) after WNT5A short-term treatment (FIGS. 6G-6H). Together, these data indicate that WNT5A activates the non-canonical PCP pathway during EP into β cell maturation.

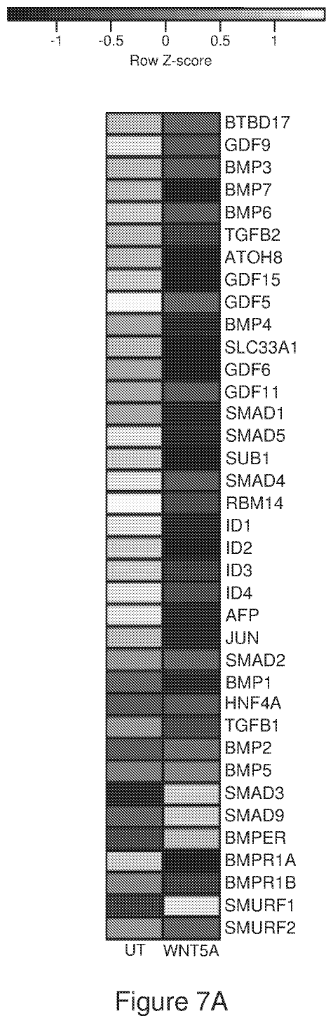

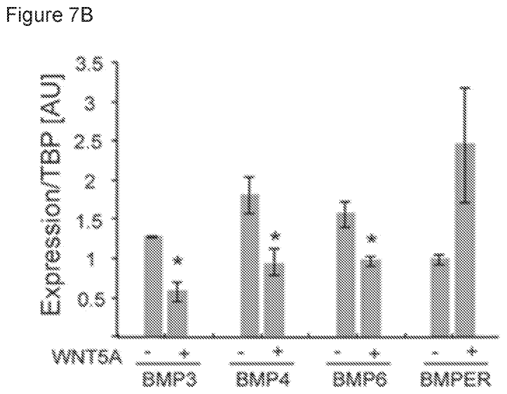

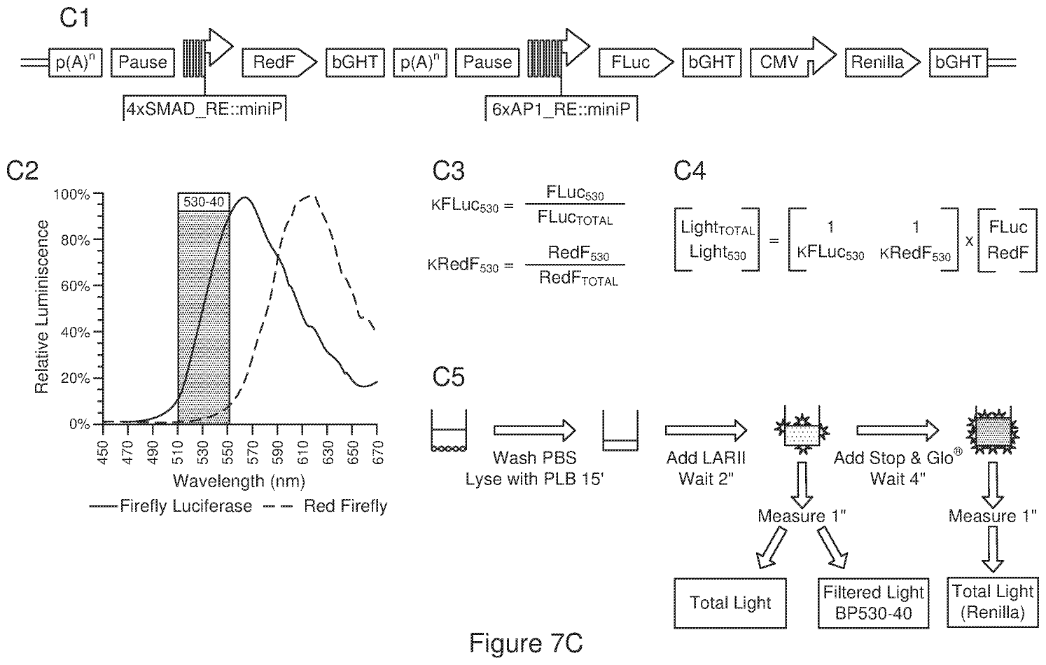

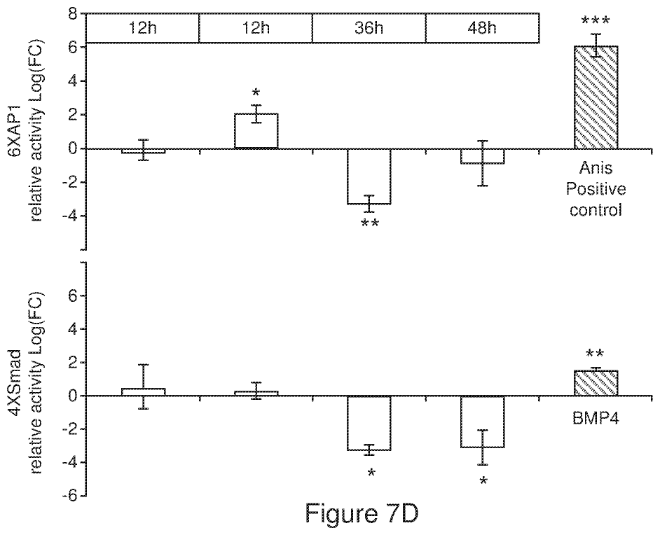

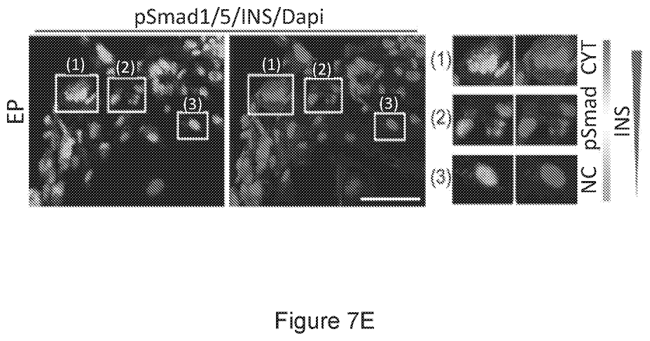

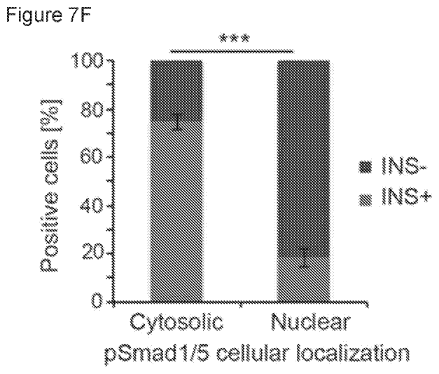

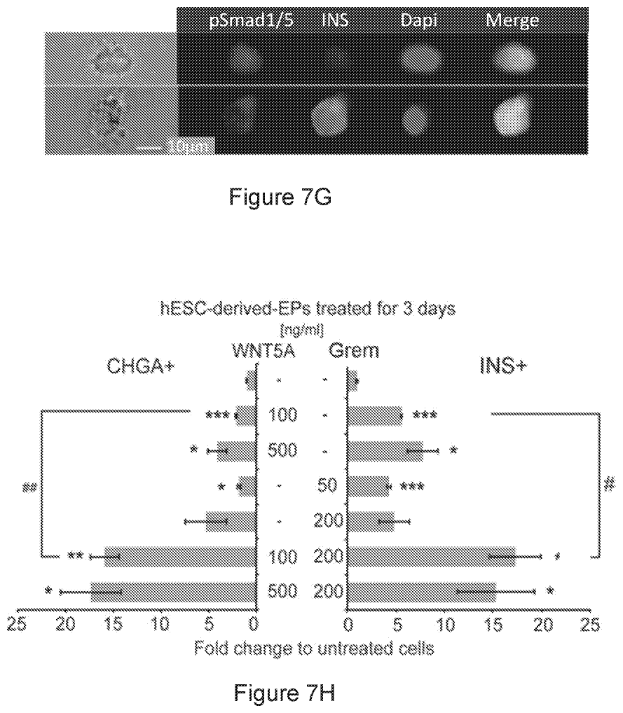

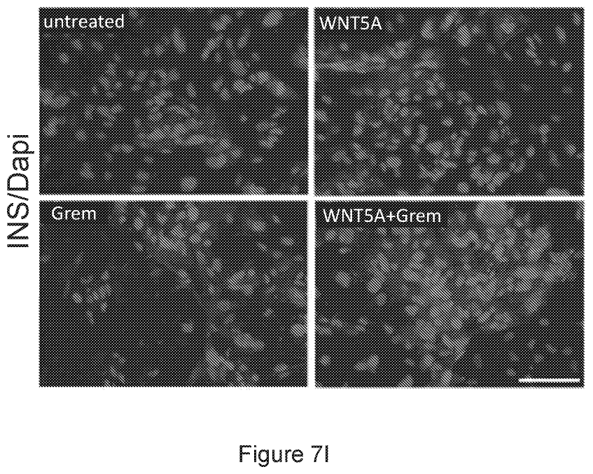

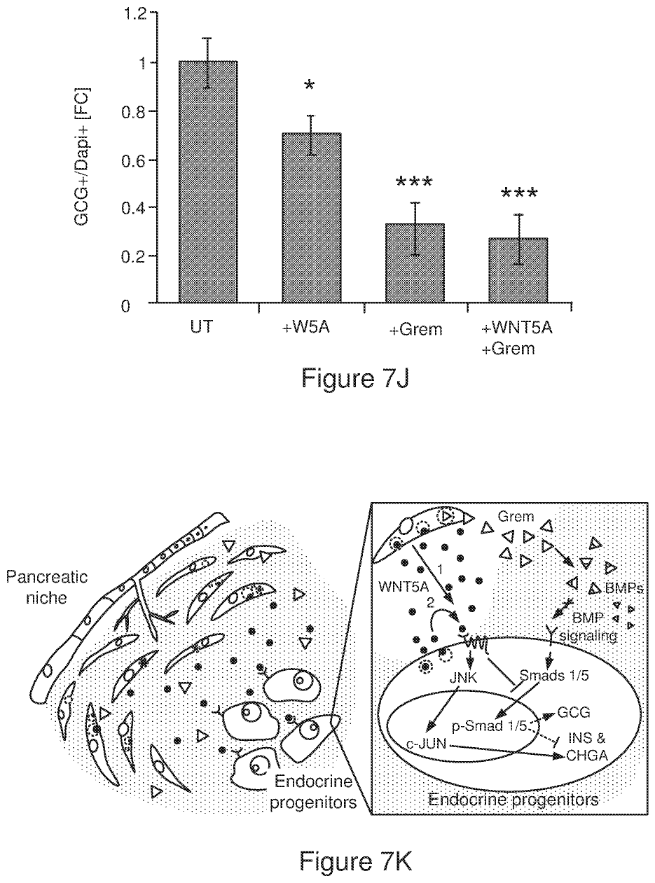

RNA-sequencing data analysis also revealed potential link between WNT5A signaling and BMP suppression during β cell differentiation (FIGS. 6A-6C and 7A), as 5-day long treatment with WNT5A in EPs downregulated BMP3, 4, and 6, as well as GDF5 and 9, but upregulated DCN and a BMP antagonist BMPER; as selectively verified by qPCR (FIGS. 6B and 7A-7B). Therefore, it was investigated whether crosstalk between the BMP and WNT5A pathways contributes to β cell formation. As a morphogen from the pancreatic mesenchyme, BMP has been shown to be crucial for pattern formation in the mesenchyme and remodeling of the vascular structure (Ahnfelt-Ronne et al., 2010), and inhibition of the Bmp receptor Alk8 in zebrafish causes PPs to preferentially differentiate into β cells (Chung et al., 2010). Studies using zebrafish, mouse embryos, and mouse ESCs have shown that Bmp signaling is essential for hepatic specification (Chung et al., 2008; Gouon-Evans et al., 2006; Rossi et al., 2001). In fact, suppressing the BMP pathway by adding Noggin has been applied in various in vitro β cell differentiation protocols, prior to and during the retinoic acid induction step, presumably to suppress hepatic development (Cai et al., 2009; Kroon et al., 2008; Mfopou et al., 2010; Zhang et al., 2009). Recently, it has been demonstrated that blocking BMP following PDX1 induction resulted in precocious INS expression, although the mechanism remains vague (Russ et al., 2015).

To determine the relationship between the BMP pathway and WNT5A, the inventors first performed a novel triple luciferase reporter assay to investigate whether WNT5A treatment caused simultaneous activation of AP-1 and down-regulation of Smad, a downstream effector of BMPs (FIGS. 7C-7D). A multigenic construct was generated that included two transcriptional to monitor two biological pathways and a third one that is used to normalize the data between biological replicates. The vector contained 4 Smad binding elements upstream of a synthetic minimal promoter to drive the expression of the Red Firefly luciferase, followed by 6 copies of the AP-1 binding element and minimal promoter driving the expression of the Firefly luciferase (FLuc). The standard reporter was Renilla luciferase and was, driven by the Cytomegalovirus enhancer and promoter. The WNT5A treatment activates AP-1 activity after 24h and decreases Smad activity at 36 and 48h (FIG. 7D).

The inventors then tested for Smad1/5 activation in hESC-derived β cells using imunofluorescence and single cell flow cytometry imaging. There was correlation between the cellular localization of phosphorylated Smad1/5 and INS expression, with nuclear vs. cytosolic Smad1/5 indicating ability vs. inability to activate the BMP pathway (FIGS. 7E-7G). About 80% of cells with high INS protein levels had pSmad1/5 localized in the cytoplasm, whereas in cells with low or no-INS expression pSmad1/5 was predominantly in the nucleus (FIGS. 7E-7G), suggesting that BMP and Smad activity might inhibit INS expression.

To mimic an in vivo source of BMP inhibition, the inventors next treated EPs with Gremlin1 (FIG. 7H), a BMP antagonist that is also upregulated in Wk17.5h and 20.1 M-E primary cells (FIG. 3G). Treatment with 200 ng/ml Gremlin1 increased both CHGA+ and INS+ cell numbers by 5-fold, while the combination of 500 ng/ml WNT5A and Gremlin1 increased CHGA+17-fold and INS+16-fold (FIGS. 7H-7I). Moreover, single WNT5A or Gremlin1 treatment or treatment with both factors, caused a 3-fold decrease in the number of GCG+ cells, which are a common by-product of β cell in vitro differentiation (FIG. 7J).

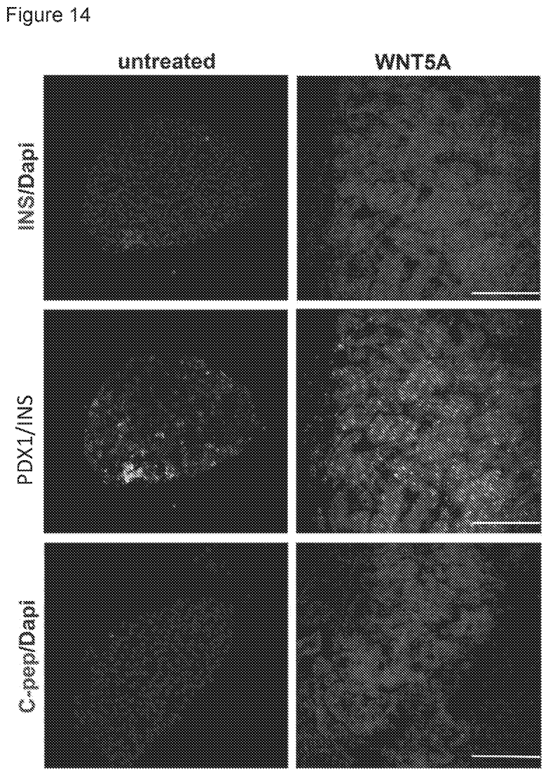

Lastly, to test whether WNT5A-treated hEPs differentiate into β cells in vivo, the inventors implanted cells into the kidney capsule of SCID-Beige mice. Immunofluorescent analysis 12 weeks after transplantation demonstrated significantly more PDX1, INS, and C-peptide positive cells in grafts of WNT5A-treated EPs, compared to untreated EPs (FIG. 14).

These results indicate that M-E cells are a source of WNT5A and BMP inhibitors. The WNT-BMP signaling crosstalk between the pancreatic niche and EPs profoundly and specifically influences EP differentiation into β cells in stage specific manner (FIG. 7K). This interaction between WNT5A and BMP led to activation of the JNK/c-Jun/AP pathway as well as upregulation of CHGA and INS while suppressing the alpha cell marker GCG.

Growth factors with the fold increase in number of ins+ cell generate from hESCs:

Endocan+SERPINF1(4.5 fold)

Endocan+WNT5A(5.5 fold)

Endocan+HGF(4 fold)

SERPINF1+WNT5A (5.5 fold)

SERPINF1+HGF(3 fold)

WNT5A+HGF (3 fold)

Endocan+SERPINF1+WNT5A(6 fold)

Endocan+SERPINF1+HGF (5 fold)

WNT5A (6 fold)

### Example 9

### Examples of Materials and Methods

Material and Methods

Derivation of Human M-E Cell Lines

To derive M-E cells, human fetal pancreas, duodenum, and spleen, were obtained from 9.1 to 20.1 weeks after fertilization, in accordance with Institutional Review Board guidelines. Each tissue was chopped into approximately 4 mm³ cubes. Samples were transferred to 6-well plates and kept at 37° C. for 10 min to allow attachment of the tissue to the plate surface. Then, DMEM:F12 media (+10% FBS, 1 xpenicillin-streptomycin, 1 xGlutamax (all Invitrogen)) was added. Over the subsequent 2 weeks, media was changed every other day, and wells were monitored for outgrowth of M-E cells. Once 50% confluent, the cubes were removed and M-E cells were trypsinized using 0.25% trypsin-EDTA and expanded until passage 3. For each stage, there were at least two independent cell line derivations completed. To derive primary murine M-E cells, ICR embryos were collected at e13.5, 14.5 and 18.5 and pancreas was processed as described above. In addition, previously established cell lines were used: human dermal fibroblasts (HDFs, ATCC), human umbilical vein endothelial cells (HUVECs, ATCC), mouse embryonic fibroblasts (mefs, E12.5 ICRs, Taconic) and mouse islet endothelial cells (MS1, ATCC).

hESC Maintenance and Pancreatic Differentiation

hESC, ISL1-EGFP Hues8 and H1, were maintained under a feeder-free system on Geltrex (Invitrogen) in TeSR-E8 media (Stemcell Technologies). Cells were passaged at 70-80% confluence using TrypLE (Invitrogen). After dissociation, cells were seeded in TeSR-E8 media+10 μM Y-27632 for 24h. Differentiation was started when cells were 90% confluent. The following media and growth factors/small molecules (see also Table 3) were used: Day1: MCDB-131 media with 0.1% BSA+10 mM glucose+ActivinA and CHIR99021. Day2-3: MCDB-131 media+0.1% BSA+10 mM glucose+ActivinA. Day4-5: MCDB-131+0.1% BSA, 10 mM glucose, VitC+KGF. Day6-9: MCDB-131 2% BSA+5.5 mM glucose+Vit.C+ITS (Invitrogen)+ActivinA+KGF+RA+SANT-1+Noggin. Day10-12: MCDB-131+2% BSA+5.5 mM glucose+VitC+ITS+SANT-1+Noggin+PdBU. Day13-15: MCDB-131+2% BSA+5.5 mM glucose+VitC+ITS+Noggin+AlK5i. EPs were dissociated and seeded as 25,000 cells/well on Geltrex-coated 96-well plates in DMEM media (Invitrogen) with B27 Supplement (Thermo Fisher Scientific), later called B27 media, supplemented with 10 M Y-27632 for 24h. Growth factors (Table 3) were added to B27 media, at two concentrations, and tested on EPs for 3 days. After 3 days, cells were PBS washed, fixed with 4% PFA/PBS for 20 min at room temperature (RT), washed twice with PBS and stained.

Co-Culture of hESC-Pancreatic Progenitors and Mesenchymal-Endothelial Cell Lines

Co-culture of M-E cells and hESC-derived progenitors was performed in three settings. The first was cell-cell interaction, the second was culture of PPs or EPs in conditional media collected from M-E cells, and the third was culture of EPs on the M-E ECM matrix. For the first assay, M-E cells were plated on 6 well-plates 24h in advance. Mitotic inactivation was performed for 2h with 10 g/ml mitomycin C (Sigma-Aldrich) followed by three washes with PBS. In the meantime, hESCs were differentiated to either the PP or EP stage and plated on M-E cells at a density of 60,000 cells per cm². As controls, mefs, laminin or gelatin-coated plates were used. Conditional M-E media was prepared from 40-80% confluent M-E cells cultured in the same media as for hESC differentiation, and media were collected every day for 6 days. The collected medium, “conditional medium” was later used as a base medium to differentiate PPs or EPs. For the third assay, M-E ECM matrix plates were prepared as follows: confluent M-E cells were cultured on 6-well plates for 6 days, after which cells were removed by short non-enzymatic, EDTA treatment, leaving the ECM matrix behind. PPs or EPs were plated on these ECM-plates and differentiated into β cells.

GSIS

Cells were washed with Krebs buffer (128 mM NaCl, 5 mM KCl, 2.7 mM CaCl₂, 1.2 mM MgCl₂, 1 mM Na₂HPO₄, 1.2 mM KH₂PO₄, 5 mM NaHCO₃, 10 mM HEPES, 0.1% BSA) and then pre-incubated in 2.8 mM D-glucose Krebs buffer for 2h. Cells were then incubated in fresh-low glucose Krebs, followed by 16.7 mM and then in Krebs buffer with 2.8 mM glucose and 30 mM KCl for 30 min at each condition. After incubation, supernatant was collected. Between incubations cells were washed 2 times with Krebs buffer. This procedure was repeated at least 3 times for different time points and coculture combinations. At the end, cells were dispersed into single cells using TrypLE Express and quantified by Countess (Invitrogen). C-peptide was measured using the Human Ultrasensitive C-peptide ELISA (Mercodia). The C-peptide amount was normalized to the cell number.

Detailed information of human islets isolation is described elsewhere herein. Human islets donor data: 67-year-old male, 44-year-old and 54-year-old female. Upon arrival, islets were seeded in 804G-coated 96-well plates and incubated in CMRL1066 media (Mediatech Inc.) supplemented with 10% human serum overnight. After 3 days, GSIS was performed.

Whole mRNA Sequencing

Total RNA was extracted as described elsewhere herein and RNA quality was assessed using 2100 Bioanalyzer (Agilent Genomics). Samples with RIN≥9 preceded to library preparation using TruSeq stranded mRNA Library Prep Kit LT (Illumina) according to the manufacturer's protocol. Library concentration was determined by qPCR (KAPA Library Quantification Kit) to pool equal amounts of libraries with different adaptor indexes. Sequencing was performed using NextSeq500 (Illumina).

Dual-Pathway Luciferase Vector and Multicolor Luciferase Assay

The dual-pathway luciferase reporter vector was generated using the GoldenBraid2.0 Assembly Platform (Sarrion-Perdigones et al., 2011; Sarrion-Perdigones et al., 2013). Briefly, transcriptional units comprising the promoter elements, the CDS of the corresponding luciferase and the bovine growth hormone terminator were first assembled, and then these were latter combined in successive rounds of assembly to build the multigenic vector used in this assay. For the multicolor luciferase assay, H1-derived EPs were first dissociated into 96-well plates using method previously described and incubated overnight to allow cell attachment. Transient transfections were then performed using 0.75 μl Lipofectamine2000 with 150 ng of dual-pathway luciferase vector for each well of the 96-well plate and incubated for 24h. Positive controls (CMV:FLuc:bGHT, CMV:RedF:bGHT and CMV:Renilla:bGHT) were transfected in separate wells to adjust the transmission constants for each luciferase. Transfected EPs were further treated with 500 ng/ml WNT5A, 200 ng/ml BMP4, 1 ng/μl Anisomycin dissolved in basal media, as previously described. At the determined harvesting point, culture media was removed and wells were washed with PBS. 35 μL of passive lysis buffer (PLB) were added to the wells. Culture plates were incubated at room temperature for 15 min on a rocking platform and stored at −80° C. for further assay until all data points were collected. After thawing the lysates, they were transferred to a 384-well plate and the luciferase assay was performed in a CLARIOstar illuminometer. 10 μL of LARII reagent was added with the built-in injectors and after 2 seconds, the total light and the BP filtered light emitted by the FLuc and RedF mixture were measured for 1 second. Finally, 15 μL of Stop & Glo® reagent were injected and after 4 seconds the emitted light by Renilla luciferase was measured. The activity corresponding to FLuc and RedF that were simultaneously measured after the LARII reagent was added were calculated according to the method proposed by Nakayima et al (Nakajima et al., 2005) that is adjusted to this particular assay and explained in FIG. 7D.

Immunofluorescent Analysis

Cells were incubated with 5% donkey serum (Jackson ImmunoResearch Laboratories) in PBST (PBS+0.1% Triton-X) for 30 min to avoid nonspecific binding of the antibody and to permeabilize the cells. Primary antibodies, diluted in 5% donkey serum in PBST, were then added and incubated at 4° C. overnight with shaking. After primary antibody incubation, cells were washed 3 times with PBST and secondary antibodies conjugated with Alexa-Fluor Dyes (Jackson ImmunoResearch Laboratories) and diluted in 5% donkey serum in PBST were added to the cells for 30 min at RT. Then, cells were thrice washed with PBST and nuclei were stained with Dapi (Roche Diagnostics). Antibody sources, catalog numbers and dilutions are listed in Table 2. For imaging, Leica DMI6000 or confocal Leica TCS SPE was used. Images were initially processed by LAS X software and then further analyzed and quantified using ImageJ software (NIH, W Rasband, http://rsb.info.nih.gov/ij) using cell counter plug-in. Typically, at least 5 randomly selected images were counted per condition.

Flow Cytometry

Cell were dissociated, washed with PBS, filtered through 40 μm cell strainer and fixed with 4% PFA for 10 min at RT. 5% donkey serum in PBST (PBS+0.2% Triton-X) was used to block unspecific binding of antibody and to permeabilize cells by 30 min incubation on ice. Primary antibodies were then added as described in Table 2 for 30 min at 4° C. with shaking. After primary antibody incubation, secondary antibodies conjugated with fluorophore, were added and incubated for 30 min at 4° C. with shaking. Then cells were centrifuged at 1500 rpm for 5 min and washed with FACS buffer (PBS+2% FBS+10 mM EDTA) twice. Stained cells were filtered through 40 m cell strainer before flow cytometry. FACS analysis was performed using LSRII (BD Biosciences) and Diva software package. For all the samples, 10,000 events were captured and FlowJo was used for gating and analysis.

Imaging Flow Cytometry to Analyze pSmad1/5 Localization

To determine cellular localization of pSmad1/5 and INS, one million of EPs was dissociated and filtered to single cell suspension as previously described and then fixed with 4% PFA/PBS with 0.1% Saponin for 30 min at 40 C. After fixation, cells were centrifuged at 3,000 g for 3 min and washed with 0.1% Saponin, 1% BSA in PBS followed by incubation with primary and then secondary antibody diluted with 0.1% Saponin, 1% BSA/PBS. ImageStreamX MarkII (Millipore) was used to capture high-resolution single cell images to detect Dapi, INS and pSMAD1/5 cellular localization. 10,000 events were acquired and compensation was adjusted to minimize spectral overlap between the fluorophores, used in the experiment, which are Dapi, Alexa488 and TRITC. Data were analyzed by IDEAS software (Millipore).

qPCR-Based Gene Expression Analysis

Total RNA was isolated using TRIzol (Thermo Fisher Scientific) according to the manufacturer's protocol. DNAase (Qiagen) treatment was performed to remove genomic DNA. cDNA was synthesized using iScript (Biorad) by using one g of RNA. For qPCR, KAPA SYBR FAST (Kapa Biosystems) and Connect CFX light cycler (Biorad) for PCR reaction (<40 cycles were used). Primers were designed using qPrimerDepot in such way that the PCR product spans across exons junction. Primer sequences are listed in Table 1. Primer specificity was checked using CFX manager software v3.1 (Applied Biosystems) and PCR product electrophoresis. Threshold data were analyzed by CFX manager software v3.1 using Comparative Ct relative quantitation method with TBP as internal control.

Microarray Analyses

50 ng of total RNA combined with RNA spike mix were reverse-transcribed using a T7 Primer Mix to produce cDNA. The cDNA product was transcribed using T7 RNA Polymerase, producing cyanine-3-labeled cRNA. The labeled cRNA was purified using a Qiagen RNeasy Mini Kit. Purified products were quantified using the NanoDop spectrophotometer for yield and dye incorporation, and tested for integrity on the Agilent Bioanalyzer. 600 ng of the labeled cRNA were fragmented. 480 ng of fragmented cRNA was loaded onto each of the Human G3 v2 8×60K Agilent Expression arrays. The arrays were hybridized in an Agilent Hybridization Chamber for 17h at 65° C. with 10 rpm rotation. The arrays were washed using the Agilent Expression Wash Buffers 1 and 2, followed by acetonitrile, as per the Agilent protocol. Once dry, the slides were scanned with the Agilent Scanner (G2565BA) using Scanner Version C and Scan Control software version A.8.3.1. Data extraction and quality assessment of the microarray data was completed using Agilent Feature Extraction Software Version 11.0.1.1. Pearson's correlation was created using Prism 6. Heatmaps were generated in R (version 3.2.3) using heatmap.2 from gplots package (version 2.17.0) with viridis (version 0.4.0), and ggbiplots (Wickham, 2009). The data is submitted at NCBI under GEO number GSE102877.

RNA-Seq Data Analysis

After preliminary analyses, showing a significant Pearson Correlation Coeficient for gene expression, two biological replicates were concatenated and analysed as single samples. This step yielded four samples, two for each treatment (5 days and 12h), as well as untreated samples. All reads were considered single-end in the bioinformatic analyses.

For Venn diagram, TFactS analysis and Gene enrichment analysis (GSEA), sequencing reads were first aligned using TopHat and the gene differential expression were assembled and analyzed using Cufflinks. Significantly up- and down regulated genes were determined by comparing Fragments Per Kilobase of transcript per Million mapped reads (FPKM) between untreated and WNT5A treated samples with p less than 0.05. Genes upregulated >2 fold and downregulated <0.5 fold were used to generated Venn diagram from BioVenn and the gene function categorization was refer from Hrvatin et al., 2013. To predict which transcription factors are responsible to the gene changes in the sequencing result, TFactS analysis was performed by inputting up and down regulation gene lists. Significantly regulated transcription factors were determined with p, e, and q<0.05, as default setting of the software. For GSEA (http://software.broadinstitute.org/gsea/index.jsp), all input files were generated through GenePattern and the analysis was performed based on the instruction from Broad institute GSEA user guide with the following parameter: phenotype labels as 5dUT versus 5dWNT5A, 1000 genes set of permutations, weighted enrichment statistic, gene sets between 15 to 500, with log 2 ratio of class as metric for ranking genes. Significantly regulated pathways have p<0.01. Data GEO submission number: GSE 90785.

WNT5A Expression and Inhibitions in EPs and HDFs

pCDNA-WNT5A plasmid was obtained from Dr. Marian Waterman (Addgene #35911). Nucleofection was used for DNA delivery. One million EPs or HDFs were dissociated with TrypLE and resuspended in 20 μl P3 solution with supplement and 1 or 2 μg of DNA. Cells were nucleofected using Amaxa-4D nucleofector (Lonza) CM113 program. After nucleofection, the inventors added pre-warmed medium to the cells and incubated them at 37° C. for 5 min before plating. The transfection efficiencies were evaluated using the pmaxGFP (Lonza): the efficiency was 17.5% and was 15.7% for EPs and HDFs, respectively. To blockWNT5A autocrine signal from EPs, 1 μg of WNT5A antibody was added to every 25,000 of EPs for three days before fixation and further immunofluorescence analysis.

Generation of WNT5A KO in Wk17.5h and Wk20.1 Cell Lines

Methods and design of WNT5A KO was described in Yang et al., 2016. Three days after the nucleofection, Wk17.5h and 20.1 cells were selected with 50 μg/ml G418 (Sigma-Aldrich) and the dosage was increased to 100 μg/ml after 2 day. After 8 days of selection, cells were cultured in DMEM+10% FBS+Glutamax+3-mercaptoethanol (Invitrogen) to evaluate efficiency of KO.

Generation of SC-β Cells and WNT5A Treatment

SC-β cells were generated using the protocol as previously described (Pagliuca et al., 2014). WNT5A were introduced into the differentiation from EP stage (EN in the original paper) together with T3, ALK5i in CMRL media for the first two days and then change into T3, ALK5i in CMRL media from the third day. Samples were collected at the 4^{th} day and 12^{th} day counting from EP stage and were fixed with 4% PFA and thrice washed with PBS for 10 min. For whole-mount staining, samples were first blocked with 5% donkey serum in PBST overnight and incubated with antibodies overnight as described above.

TOPFLASH Reporter Assay

For TOPFLASH reporter assay, 80,000 EPs were transfected with 0.5 μg of TOPFLASH (Addgene #12456) or FOPFLASH (Addgene #12457) from Dr. Randall Moon, together with 0.25 μg pRLTK using Lipofectamine 2000 (Invitrogen) for 48h. Cells were then treated with CHIR99021 (as positive control), DMSO (mock control) or WNT5A for 3 days before collecting the samples. Luciferase assay was performed using Dual luciferase assay system (Promega) which Luciferase and Renilla signal were measured by TD20/20 Luminometer (Turner designs).

Protein Extraction and Western Blotting for Phosphorylated JNK and Total JNK

ISL1-EGFP hESCs were differentiated into EPs and first balanced with basal media (DMEM with 1% BSA and NEAA) for 6h and then treated with 500 ng/ml WNT5A in basal media. After 12h, cell lysates were collected as imilion of EPs were pelleted, PBS washed and resuspended in 250 μl of lysis buffer (10 mM HEPES pH7.5, 10 mM MgCl₂, 5 mM KCl, 0.1 mM EDTA pH8, 0.1% TritonX-100, 0.2 mM PMSF, 1 mM DTT, 1 tab of Complete Protease Inhibitor Cocktail (Roche)). Cell lysates were centrifuged 12,000 g at 4° C. for 15 min and their supernatants were collected. BCA assay were performed using Pierce BCA protein assay kit (Thermo) to determine protein concentration. For Western blot, 30 g of protein were denatured with 4x Laemmeli buffer (40% glycerol, 8% SDS, 240 mM Tris-HCl pH6.8, 5% β-mercaptoethanol, 12.5 mM EDTA, 0.04% bromophenol blue) at 95° C. 3 min and resolved in 8% SDS-PAGE. PVDF membranes (BioRad) were used for transfer and membrane were blocked with 5% BSA in Tris-buffered saline with 1% Tween 20 (TBST) for 1h before applying primary antibody. Membranes were washed thrice for 10 min and anti-rabbit IgG-HRP (GE Life Science) were added for 3h at RT, followed by three washes. HyGLO™ Quick Spray Chemiluminescent HRP Antibody Detection Reagent (Denville Scientific Inc.) was used to detect antigen and the membrane was developed using CL-XPosure Film (Thermo Scientific). Membrane stripping was performed using mild stripping buffer (200 mM glycine, 0.1% SDS, 1% Tween20, pH 2.2) according to the Abcam's instructions.

Human Islet Isolation

Human pancreata were obtained with informed consent for transplant or research use from relatives of heart-beating, cadaveric, multi-organ donors through the efforts of The National Disease Research Interchange (NDRI), Tennessee Donor Services, the Mid-South Transplant Foundation, and the United Network for Organ Sharing. Donor demographics were collected at the time of acceptance and included age in years, gender, race, body mass index, history of alcohol intake, and history of hypertension. Donor-related laboratory data included donor blood glucose, serum amylase, lipase, liver function tests (ALT, AST), cytomegalovirus infection status, and procurement and preservation parameters such as pancreatic warm and cold ischemia times, ventilation time, pancreas weight and adequacy of pancreas perfusion were also recorded. All pancreata in this study were perfused using University of Wisconsin (UW) solution. Human islets were isolated from cadaver donors using an adaptation of the automated method described by Ricordi et al. (Ricordi et al., 1988). Liberase (Boehringer Mannheim, Indianapolis, Ind.) was the enzyme used in all the isolations in this study. Liberase was dissolved in cold (4° C.) Hank's balanced salt solution (HBSS) (Mediatech, Inc., Herndon, Va.) that was supplemented with 0.2 mg/ml Dnase (Sigma Chemical Co., St. Louis, Mo.), 1% penicillin-streptomycin (Sigma Chemical Co.), 20 mg/dl calcium chloride (J.T. Baker, Inc., Phillipsburg, N.J.), and HEPES (Sigma Chemical Co.). Once dissolved, the pH was adjusted to between 7.7 and 7.9. The enzyme preparation was then sterile filtered, warmed to 37° C., and used for the intraductal distension of the pancreas. The distended pancreas was cut into several pieces, placed in the Ricordi's chamber, and the Heating circuit started. Pancreatic digestion was performed at 37° C. until more than 90% free islets were observed in the sample. Digested tissue was collected into cold HBSS supplemented with 20% human serum and 1% penicillin-streptomycin solution and centrifuged at 400 g at 4° C. for 5 min. Tissue pellets were pooled into cold UW solution and held at 4° C. for 1h with periodic mixing. Islet purification was performed on a COBE 2991 Cell Processor (COBE BCT, Lakewood, Colo.) using OptiPrep (Nycomed Pharma AS, Oslo, Norway) as a step-gradient based on a modification of the procedure of London et al (Robertson et al., 1993). Islet culture: Aliquots from human islet isolations were cultured in SFM containing 1% ITS, 1% L-glutamine (Life Technologies, Gaithersburg, Md.), 1% antibiotic antimycotic solution (Sigma Chemical Co.), and 16.8 μM/L zinc sulfate. ITS (1%; Collaborative Biomedical Products, Bedford, Mass.) as described (Fraga et al., 1998). Human islets donor data: 67-year-old male, and 54-year-old female. For wholemount immunofluorescence staining, islets were first fixed 15 min in 4% PFA at room temperature shaker, followed by three 30 min washes in PBS. Islets were incubated at 4° C. overnight rotor for the blocking, primary and secondary incubation, as described before.

Cell Migration Assays:

a) Scratch Assay.

EPs were dissociated and seeded into 12 well-plate to reach 90% confluence before a scratch was made. 200 μl pipette tip was used to create a smooth scratch and followed by 3h of mitomycin C treatment. Cells were then washed thrice with PBS and B27 media with or without WNT5A was added. Pictures of the scratch wound were taken at 0, 6, 12, 18, 24 and 30h after scratch to observe cell migration. Pictures were analyzed by counting cells migrating into the gap using ImageJ.

b) Transwell Assays.

Transwell assays were performed using FluoroBlock Insert, 8 μM pore size (Corning). The bottom 24 wells and the insert of transwell were coated with Geltrex to retained cells. At the experiment day, 1.5×10⁵ hESC-derived EPs were seeded in each well at the transwell insert in B27 media with 10 μM Y-27632 and the well bottoms were replenished with either B27 media as control, B27 media+100 or 500 ng/ml WNT5A, or conditional media from Wk9.1, 17.5h and 20.1. After one week, the cell attached to the bottom wells were stained with Dapi and were counted.

In Vivo Transplantation

WNT5A treated hESC-derived EPs were treated with 500 ng/ml WNT5A for 3 days and cultured in B27 for 11 days before transplantation. Control EPs were maintained in B27 for 2 weeks. 1.5 million of hESC-derived EPs were mixed with equal volumes of matrigel and injected into the kidney capsule into 6 week-old SCID-Beige mice (Taconic Bioscience). For control N=6 and WNT5A treated N=8 in 2 cohorts. Grafts were collected at X weeks, washed in PBS, and fixed with 4% PFA for 1h. After fixation, tissues were washed with PBS and incubated with 30% sucrose/PBS overnight before embedding into OCT for cryosectioning. All animal experiments were approved by Baylor College of Medicine Institutional Animal Care and Use Committee.

TABLE 1
List of qPCR primers.

| Gene | Primer sequence |
|---|---|
| Vimentin | Tgcaggctcagattcaggaa (SEQ ID NO: 1) |
| | ctccggtactcagtggactc (SEQ ID NO: 2) |
| PECAM1 | tcccctaagaattgctgcca (SEQ ID NO: 3) |
| | ttcttcccaacacgccaatg (SEQ ID NO: 4) |
| FSP1 | aggggtgaagaagatgggtg (SEQ ID NO: 5) |
| | ccagtcacaccagcaatcac (SEQ ID NO: 6) |
| FLK1 | ttacttgcaggggacagagg (SEQ ID NO: 7) |
| | ttcccggtagaagcacttgt (SEQ ID NO: 8) |
| VE | taccaggacgctacaccat (SEQ ID NO: 9) |
| CADHERIN | aaaggctgctggaaaatggg (SEQ ID NO: 10) |
| ICAM | agagaccccgttgcctaaaa (SEQ ID NO: 11) |
| | cagtacacggtgaggaaggt (SEQ ID NO: 12) |
| VWF | tgcaacacttgtgtctgtcg (SEQ ID NO: 13) |
| | cgaaaggtcccagggttact (SEQ ID NO: 14) |
| INS | agcctttgtgaaccaacacc (SEQ ID NO: 15) |
| | gctggtagagggagcagatg (SEQ ID NO: 16) |
| PDX1 | aagtctaccaaagctcacgcg (SEQ ID NO: 17) |
| | gtaggcgccgcctgc (SEQ ID NO: 18) |
| WNT5A | ctccgctcggattcctc (SEQ ID NO: 19) |
| | caaagcaactcctgggctta (SEQ ID NO: 20) |
| TBP | tgtgcacaggagccaagagt (SEQ ID NO: 21) |
| | attacttgctgccagtctgg (SEQ ID NO: 22) |
| GCG | aagcatttactagtggctggatt (SEQ ID NO: 23) |
| | tgatctggatttctcctctgtgtct |
| | (SEQ ID NO: 24) |
| BMP3 | cagaaatacagtgtggcagaca (SEQ ID NO: 25) |
| | acacggttcgcagctac (SEQ ID NO: 26) |
| BMP4 | ctcctagcaggacttggcat (SEQ ID NO: 27) |
| | tggctgtcaagaatcatgga (SEQ ID NO: 28) |
| BMP6 | tgcaggaagcatgagctg (SEQ ID NO: 29) |
| | gtgcgttgagtgggaagg (SEQ ID NO: 30) |
| BMPER | ggacaggagagaatgggaca (SEQ ID NO: 31) |
| | tgtgtttgagggtgtgcagt (SEQ ID NO: 32) |
| FZD3 | tgccaactatgagagccatc (SEQ ID NO: 33) |
| | caacgtggatacaagaacgc (SEQ ID NO: 34) |
| ONECUT1 | tttttgggtgtgttgcctct (SEQ ID NO: 35) |
| | agaccttccggaggatgtg (SEQ ID NO: 36) |
| PCSK2 | tttcggtcaaatccttcctg (SEQ ID NO: 37) |
| | tgcaaaggccaagagaagac (SEQ ID NO: 38) |
| SOX9 | gtggtccttcttgtgctgc (SEQ ID NO: 39) |
| | gtacccgcacttgcacaac (SEQ ID NO: 40) |
| FOXA2 | catgttgctcacggaggagt (SEQ ID NO: 41) |
| | tttaaactgccatgcactcg (SEQ ID NO: 42) |

TABLE 2
List of antibodies

| Antibody | Manufacturer | Catalog number | Dilution |
|---|---|---|---|
| Ins | Dako | A056401 | 1:100 |
| Glucagon | Santa Cruz | Sc-7779 | 1:100 |
| Chromogranin A | Abcam | Ab15160 | 1:100 |
| C-peptide | DSHB | GN104-s | 1:100 |
| GFP | Abcam | Ab13970 | 1:1000 |
| PECAM1 | DSHB | P2B1-c | 1:100 |
| Vimentin | Millipore | Ab5733 | 1:1000 |
| WNT5A | Santa Cruz | Sc-23698 | 1:100 |
| PDX1 | R&D | AF2419 | 1:100 |
| NKX6.1 | DSHB | F64A6B4 | 1:100 |
| pH3 | Millipore | 06570 | 1:100 |
| FZD3 | Gift from Dr. Jeremy | | 1:100 |
| | Nathans | | |
| p-JNK | Cell signaling | 4668 | 1:1000 |
| JNK | Cell signaling | 9252 | 1:1000 |
| p-c-JUN | Cell signaling | 9261 | 1:1000 |
| p-Smad1/5 | Cell signaling | 9516 | 1:100 |
| Beta actin | Sigma-Aldrich | A5441 | 1:5000 |

TABLE 3
Growth factors and small molecules used
in FIGS. 4-7 and FIGS. 8, 9, and 11.

| Growth | | | | | |
|---|---|---|---|---|---|
| factors | Manufacturer | Concentration 1 | | Concentration 2 | |
| Y-27632 | Stemgent | 10 | μM | | |
| Activin A | R&D | 100 ng/ml (Day 1-3 | | 20 ng/ml (Day 4-9 | |
| | | of pancreatic | | of pancreatic | |
| | | differentiation) | | differentiation) | |
| CHIR99021 | Stemgent | 3 | μM | | |
| Ascorbic | Sigma-Aldrich | 44 | mg/l | | |
| acid | | | | | |
| KGF | Peprotech | 12.5 | ng/ml | | |
| RA | Sigma-Aldrich | 2 | μM | | |
| SANT-1 | Sigma-Aldrich | 0.25 | μM | | |
| Noggin | R&D | 100 | ng/ml | | |
| PdBU | Sigma-Aldrich | 1 | μM | | |
| AlK5i | Axxora | 1 | μM | | |
| FGF7 | Peprotech | 50 | ng/ml | 100 | ng/ml |
| HGF | R&D | 50 | ng/ml | 100 | ng/ml |
| PDPN | R&D | 100 | ng/ml | 500 | ng/ml |
| SERPINF1 | R&D | 500 | ng/ml | 1 | μg/ml |
| WNT5A | R&D | 100 | ng/ml | 500 | ng/ml |
| LIF | Home-made | ~1 | U/ml | ~0.5 | U/ml |
| EGF | R&D | 50 | ng/ml | 25 | ng/ml |
| THBS2 | R&D | 5 | μg/ml | 1 | μg/ml |
| IGF1 | R&D | 10 | ng/ml | 50 | ng/ml |
| Endocan | R&D | 1 | ng/ml | 5 | ng/ml |
| WNT3A | R&D | 20 | ng/ml | 40 | ng/ml |
| Gremlin1 | R&D | 50 | ng/ml | 200 | ng/ml |
| SP600125 | EMD Millipore | 80 | μM | 160 | μM |
| BMP4 | R&D | 200 | ng/ml | | |
| Anisomycin | Sigma | 1 | ng/μl | | |

TABLE 4
Growth factors combinations used in FIG. 9.

| Growth | | |
|---|---|---|
| Factors | Concentration 1 | Concentration 2 |
| E + S | 1 ng/ml Endocan + 500 ng/ml SERPINF1 | 1 ng/ml Endocan + 1 μg/ml SERPINF1 |
| E + W | 1 ng/ml Endocan + 100 ng/ml WNT5A | 1 ng/ml Endocan + 500 ng/ml WNT5A |
| E + H | 1 ng/ml Endocan + 50 ng/ml HGF | 1 ng/ml Endocan + 100 ng/ml HGF |
| S + W | 1 μg/ml SERPINF1 + 100 ng/ml WNT5A | 1 μg/ml SERPINF1 + 500 ng/ml WNT5A |
| S + H | 1 μg/ml SERPINF1 + 50 ng/ml HGF | 1 μg/ml SERPINF1 + 100 ng/ml HGF |
| W + H | 100 ng/ml WNT5A + 50 ng/ml HGF | 500 ng/ml WNT5A + 50 ng/ml HGF |
| E + S + W | 1 ng/ml Endocan + 1 μg/ml SERPINF1 + | 1 ng/ml Endocan + 1 μg/ml SERPINF1 + |
| | 100 ng/ml WNT5A | 500 ng/ml WNT5A |
| E + W + H | 1 ng/ml Endocan + 100 ng/ml WNT5A+ | 1 ng/ml Endocan + 100 ng/ml WNT5A + |
| | 50 ng/ml HGF | 100 ng/ml HGF |

### REFERENCES

All patents and publications mentioned in this specification are indicative of the level of those skilled in the art to which the invention pertains. All patents and publications herein are incorporated by reference to the same extent as if each individual publication was specifically and individually indicated to be incorporated by reference in their entirety.
Ahnfelt-Ronne, J., Ravassard, P., Pardanaud-Glavieux, C., Scharfmann, R., and Serup, P. (2010). Mesenchymal bone morphogenetic protein signaling is required for normal pancreas development. Diabetes 59, 1948-1956.
Albelda, S., Muller, W., Buck, C., and Newman, P. (1991). Molecular and cellular properties of PECAM-1 (endoCAM:CD31)—A novel vascular cell-cell adhesion molecule. J Cell Biol 114, 1059-1068.
Arbiser, J. L., Larsson, H., Claesson-Welsh, L., Bai, X., LaMontagne, K., Weiss, S. W., Soker, S., Flynn, E., and Brown, L. F. (2000). Overexpression of VEGF 121 in Immortalized Endothelial Cells Causes Conversion to Slowly Growing Angiosarcoma and High Level Expression of the VEGF Receptors VEGFR-1 and VEGFR-2 in vivo. The American Journal of Pathology 156, 1469-1476.
Bader, E., Migliorini, A., Gegg, M., Moruzzi, N., Gerdes, J., Roscioni, S. S., Bakhti, M., Brandl, E., Irmler, M., Beckers, J., et al. (2016). Identification of proliferative and mature beta-cells in the islets of Langerhans. Nature 535, 430-434.
Ben-Shushan, E., Marshak, S., Shoshkes, M., Cerasi, E., and Melloul, D. (2001). A pancreatic beta-cell-specific enhancer in the human PDX-1 gene is regulated by hepatocyte nuclear factor 3beta (HNF-3beta), HNF-lalpha, and SPs transcription factors. The Journal of biological chemistry 276, 17533-17540.
Bhushan, A., Itoh, N., Kato, S., Thiery, J. P., Czernichow, P., Bellusci, S., and Scharfmann, R. (2001). Fgfl0 is essential for maintaining the proliferative capacity of epithelial progenitor cells during early pancreatic organogenesis. Development 128, 5109-5117.
Bilkovski, R., Schulte, D. M., Oberhauser, F., Gomolka, M., Udelhoven, M., Hettich, M. M., Roth, B., Heidenreich, A., Gutschow, C., Krone, W., et al. (2010). Role of WNT-5a in the determination of human mesenchymal stem cells into preadipocytes. The Journal of biological chemistry 285, 6170-6178.
Borowiak, M. (2010). The new generation of beta-cells: replication, stem cell differentiation, and the role of small molecules. The review of diabetic studies: RDS 7, 93-104.
Boyden, S. (1962). The chemotactic effect of mixtures of antibody and antigen on polymorphonuclear leucocytes. J Exp Med 115, 453-466.
Breier, G., Breviario, F., Caveda, L., Berthier, R., Schniirch, H., Gotsch, U., Vestweber, D., Risau, W., and Dejana, E. (1996). Molecular cloning and expression of murine vascular endothelial-cadherin in early stage development of cardiovascular system. Blood 15, 630-641.
Breton-Romero, R., Feng, B., Holbrook, M., Farb, M. G., Fetterman, J. L., Linder, E. A., Berk, B. D., Masaki, N., Weisbrod, R. M., Inagaki, E., et al. (2016). Endothelial Dysfunction in Human Diabetes Is Mediated by Wnt5a-JNK Signaling. Arteriosclerosis, thrombosis, and vascular biology 36, 561-569.
Bu, L., Jiang, X., Martin-Puig, S., Caron, L., Zhu, S., Shao, Y., Roberts, D. J., Huang, P. L., Domian, I. J., and Chien, K. R. (2009). Human ISL1 heart progenitors generate diverse multipotent cardiovascular cell lineages. Nature 460, 113-117.
Cheng, X., Ying, L., Lu, L., Galvao, A. M., Mills, J. A., Lin, H. C., Kotton, D. N., Shen, S. S., Nostro, M. C., Choi, J. K., et al. (2012). Self-renewing endodermal progenitor lines generated from human pluripotent stem cells. Cell stem cell 10, 371-384.
Chung, W. S., Andersson, O., Row, R., Kimelman, D., and Stainier, D. Y. (2010). Suppression of Alk8-mediated Bmp signaling cell-autonomously induces pancreatic beta-cells in zebrafish. Proceedings of the National Academy of Sciences of the United States of America 107, 1142-1147.
Cortijo, C., Gouzi, M., Tissir, F., and Grapin-Botton, A. (2012). Planar cell polarity controls pancreatic beta cell differentiation and glucose homeostasis. Cell reports 2, 1593-1606.
Cryer, P. E. (2014). Glycemic goals in diabetes: trade-off between glycemic control and iatrogenic hypoglycemia. Diabetes 63, 2188-2195.
D'Amour, K. A., Bang, A. G., Eliazer, S., Kelly, O. G., Agulnick, A. D., Smart, N. G., Moorman, M. A., Kroon, E., Carpenter, M. K., and Baetge, E. E. (2006). Production of pancreatic hormone-expressing endocrine cells from human embryonic stem cells. Nature biotechnology 24, 1392-1401.
Deutsch, G., Jung, J., Zheng, M., Lóra, J., and Zaret, K. (2001). A bipotential precursor population for pancreas and liver within the embryonic endoderm. Development 128, 871-881.
Dichmann, D. S., Miller, C. P., Jensen, J., Scott Heller, R., and Serup, P. (2003). Expression and misexpression of members of the FGF and TGFbeta families of growth factors in the developing mouse pancreas. Developmental dynamics: an official publication of the American Association of Anatomists 226, 663-674.
Dorrell, C., Schug, J., Canaday, P. S., Russ, H. A., Tarlow, B. D., Grompe, M. T., Horton, T., Hebrok, M., Streeter, P. R., Kaestner, K. H., et al. (2016). Human islets contain four distinct subtypes of beta cells. Nature communications 7, 11756.
Durieu-Trautmann, O., Chaverot, N., Cazaubon, S., Strosberg, A., and Couraud, P. (1994). Intercellular Adhesion Molecule 1 Activation Induces 'Qrrosine Phosphorylation of the Cytoskeleton-associated Protein Cortactin in Brain Microvessel Endothelial Cells. The Journal of biological chemistry 269, 12536-12540.
Forbes, J. M., and Cooper, M. E. (2013). Mechanisms of diabetic complications. Physiological reviews 93, 137-188.
Ford, C. E., Punnia-Moorthy, G., Henry, C. E., Llamosas, E., Nixdorf, S., Olivier, J., Caduff, R., Ward, R. L., and Heinzelmann-Schwarz, V. (2014). The non-canonical Wnt ligand, Wnt5a, is upregulated and associated with epithelial to mesenchymal transition in epithelial ovarian cancer. Gynecologic oncology 134, 338-345.
Franke, W., Grund, C., Kuhn, C., Jackson, B., and Illmensee, K. (1982). Formation of Cytoskeletal Elements During Mouse Embryogenesis: III. Primary Mesenchymal Cells and the First Appearance of Vimentin Filaments. Differentiation 23, 43-59.
Franke, W., Schmid, E., Osborn, M., and Weber, K. (1978). Different intermediate-sized filaments distinguished by immunofluorescence microscopy. Proc Natl Acad Sci USA 75.
Fuentealba, L. C., Eivers, E., Ikeda, A., Hurtado, C., Kuroda, H., Pera, E. M., and De Robertis, E. M. (2007). Integrating patterning signals: Wnt/GSK3 regulates the duration of the BMP/Smad1 signal. Cell 131, 980-993.
Harada, J., Kokura, K., Kanei-Ishii, C., Nomura, T., Khan, M. M., Kim, Y., and Ishii, S. (2003). Requirement of the co-repressor homeodomain-interacting protein kinase 2 for ski-mediated inhibition of bone morphogenetic protein-induced transcriptional activation. The Journal of biological chemistry 278, 38998-39005.
Heller, R. S., Klein, T., Ling, Z., Heimberg, H., Katoh, M., Madsen, O. D., and Serup, P. (2003). Expression of Wnt, Frizzled, sFRP, and DKK genes in adult human pancreas. Gene expression 11, 141-147.
Hofmann, T., Stollberg, N., Schmitz, M., and Will, H. (2003). HIPK2 regulates transforming growth factor-beta-induced c-Jun NH(2)-terminal kinase activation and apoptosis in human hepatoma cells. Cancer Res 63, 8271-8277.
Home, P., Riddle, M., Cefalu, W. T., Bailey, C. J., Bretzel, R. G., Del Prato, S., Leroith, D., Schernthaner, G., van Gaal, L., and Raz, I. (2014). Insulin therapy in people with type 2 diabetes: opportunities and challenges? Diabetes care 37, 1499-1508.
Jacquemin, P., Yoshitomi, H., Kashima, Y., Rousseau, G. G., Lemaigre, F. P., and Zaret, K. S. (2006). An endothelial-mesenchymal relay pathway regulates early phases of pancreas development. Developmental biology 290, 189-199.
Jaffe, E., Nachman, R., Becker, C., and Minick, C. (1973). Culture of Human Endothelial Cells Derived from Umbilical Veins. J Clin Invest 52, 2745-2756.
Jiang, F. X., Cram, D. S., DeAizpurua, H. J., and Harrison, L. C. (1999). Laminin-1 promotes differentiation of fetal mouse pancreatic beta-cells. Diabetes 48, 722-730.
Jones, T., Kao, K., Pizzo, S., and Bigner, D. (1981). Endothelial cell surface expression and binding of factor VIII:von Willebrand factor. Am J Pathol 103, 304-308.
Kesavan, G., Lieven, O., Mamidi, A., Ohlin, Z. L., Johansson, J. K., Li, W. C., Lommel, S., Greiner, T. U., and Semb, H. (2014). Cdc42/N-WASP signaling links actin dynamics to pancreatic beta cell delamination and differentiation. Development 141, 685-696.
Kesavan, G., Sand, F. W., Greiner, T. U., Johansson, J. K., Kobberup, S., Wu, X., Brakebusch, C., and Semb, H. (2009). Cdc42-mediated tubulogenesis controls cell specification. Cell 139, 791-801.
Kikuchi, A., Yamamoto, H., Sato, A., and Matsumoto, S. (2012). Wnt5a: its signalling, functions and implication in diseases. Acta physiologica 204, 17-33.
Kim, H. J., Schleiffarth, J. R., Jessurun, J., Sumanas, S., Petryk, A., Lin, S., and Ekker, S. C. (2005). Wnt5 signaling in vertebrate pancreas development. BMC biology 3, 23.
Kobberup, S., Schmerr, M., Dang, M. L., Nyeng, P., Jensen, J. N., MacDonald, R. J., and Jensen, J. (2010). Conditional control of the differentiation competence of pancreatic endocrine and ductal cells by Fgfl0. Mechanisms of development 127, 220-234.
Kroon, E., Martinson, L. A., Kadoya, K., Bang, A. G., Kelly, O. G., Eliazer, S., Young, H., Richardson, M., Smart, N. G., Cunningham, J., et al. (2008). Pancreatic endoderm derived from human embryonic stem cells generates glucose-responsive insulin-secreting cells in vivo. Nature biotechnology 26, 443-452.
Lammert, E., Cleaver, O., and Melton, D. (2001). Induction of pancreatic differentiation by signals from blood vessels. Science 294, 564-567.
Lawlor, N., George, J., Bolisetty, M., Kursawe, R., Sun, L., Sivakamasundari, V., Kycia, I., Robson, P., and Stitzel, M. L. (2017). Single-cell transcriptomes identify human islet cell signatures and reveal cell-type-specific expression changes in type 2 diabetes. Genome research 27, 208-222.
Lawson, C., and Wolf, S. (2009). ICAM-1 signaling in endothelial cells. Pharmacol Rep 61, 22-32.
Lioubinski, O., Muller, M., Wegner, M., and Sander, M. (2003). Expression of Sox transcription factors in the developing mouse pancreas. Developmental dynamics: an official publication of the American Association of Anatomists 227, 402-408.
Lyon, J., Manning Fox, J. E., Spigelman, A. F., Kim, R., Smith, N., O'Gorman, D., Kin, T., Shapiro, A. M., Rajotte, R. V., and MacDonald, P. E. (2016). Research-Focused Isolation of Human Islets From Donors With and Without Diabetes at the Alberta Diabetes Institute IsletCore. Endocrinology 157, 560-569.
Magenheim, J., Ilovich, O., Lazarus, A., Klochendler, A., Ziv, O., Werman, R., Hija, A., Cleaver, O., Mishani, E., Keshet, E., et al. (2011). Blood vessels restrain pancreas branching, differentiation and growth. Development 138, 4743-4752.
Martin, M., Gallego-Llamas, J., Ribes, V., Kedinger, M., Niederreither, K., Chambon, P., Dolle, P., and Gradwohl, G. (2005). Dorsal pancreas agenesis in retinoic acid-deficient Raldh2 mutant mice. Developmental biology 284, 399-411.
Mikels, A. J., and Nusse, R. (2006). Purified Wnt5a protein activates or inhibits beta-catenin-TCF signaling depending on receptor context. PLoS biology 4, el 15.
Negi, S., Jetha, A., Aikin, R., Hasilo, C., Sladek, R., and Paraskevas, S. (2012). Analysis of beta-cell gene expression reveals inflammatory signaling and evidence of dedifferentiation following human islet isolation and culture. PloS one 7, e30415.
Nostro, M. C., and Keller, G. (2012). Generation of beta cells from human pluripotent stem cells: Potential for regenerative medicine. Seminars in cell & developmental biology 23, 701-710.
Nostro, M. C., Sarangi, F., Yang, C., Holland, A., Elefanty, A. G., Stanley, E. G., Greiner, D. L., and Keller, G. (2015). Efficient generation of NKX6-1+ pancreatic progenitors from multiple human pluripotent stem cell lines. Stem cell reports 4, 591-604.
Ohlsson, H., Karlsson, K., and Edlund, T. (1993). IPF1, a homeodomain-containing transactivator of the insulin gene. The EMBO journal 12, 4251-4259.
Olokoba, A. B., Obateru, O. A., and Olokoba, L. B. (2012). Type 2 diabetes mellitus: a review of current trends. Oman Med J 27, 269-273.
Pagliuca, F. W., Millman, J. R., Gurtler, M., Segel, M., Van Dervort, A., Ryu, J. H., Peterson, Q. P., Greiner, D., and Melton, D. A. (2014). Generation of functional human pancreatic beta cells in vitro. Cell 159, 428-439.
Piper, K. (2004). Beta cell differentiation during early human pancreas development. Journal of Endocrinology 181, 11-23.
Piper, K., Ball*, S. G., Keeling, J. W., Mansoor, S., Wilson, D. I., and Hanley, N. A. (2002). Novel SOX9 expression during human pancreas development correlates to abnormalities in Campomelic dysplasia. Mechanisms of Development 116, 223-226.
Qu, Y., Huang, Y., Feng, J., Alvarez-Bolado, G., Grove, E. A., Yang, Y., Tissir, F., Zhou, L., and Goffinet, A. M. (2014). Genetic evidence that Celsr3 and Celsr2, together with Fzd3, regulate forebrain wiring in a Vangl-independent manner. Proceedings of the National Academy of Sciences of the United States of America 111, E2996-3004.
Rezania, A., Bruin, J. E., Arora, P., Rubin, A., Batushansky, I., Asadi, A., O'Dwyer, S., Quiskamp, N., Mojibian, M., Albrecht, T., et al. (2014). Reversal of diabetes with insulin-producing cells derived in vitro from human pluripotent stem cells. Nature biotechnology 32, 1121-1133.
Russ, H. A., and Hebrok, M. (2014). Taming the young and restless—epigenetic gene regulation in pancreas and beta-cell precursors. The EMBO journal 33, 2135-2136.
Russ, H. A., Landsman, L., Moss, C. L., Higdon, R., Greer, R. L., Kaihara, K., Salamon, R., Kolker, E., and Hebrok, M. (2016). Dynamic Proteomic Analysis of Pancreatic Mesenchyme Reveals Novel Factors That Enhance Human Embryonic Stem Cell to Pancreatic Cell Differentiation. Stem cells international 2016, 6183562.
Russ, H. A., Parent, A. V., Ringler, J. J., Hennings, T. G., Nair, G. G., Shveygert, M., Guo, T., Puri, S., Haataja, L., Cirulli, V., et al. (2015). Controlled induction of human pancreatic progenitors produces functional beta-like cells in vitro. The EMBO journal 34, 1759-1772.
Sarrion-Perdigones, A., Falconi, E. E., Zandalinas, S. I., Juarez, P., Fernandez-del-Carmen, A., Granell, A., and Orzaez, D. (2011). GoldenBraid: an iterative cloning system for standardized assembly of reusable genetic modules. PloS one 6, e21622.
Sarrion-Perdigones, A., Vazquez-Vilar, M., Palaci, J., Castelijns, B., Forment, J., Ziarsolo, P., Blanca, J., Granell, A., and Orzaez, D. (2013). GoldenBraid 2.0: a comprehensive DNA assembly framework for plant synthetic biology. Plant physiology 162, 1618-1631.
Sneddon, J. B., Borowiak, M., and Melton, D. A. (2012). Self-renewal of embryonic-stem-cell-derived progenitors by organ-matched mesenchyme. Nature 491, 765-768.
Stahlman, M. T., Gray, M. E., and Whitsett, J. A. (1998). Temporal-spatial distribution of hepatocyte nuclear factor-3beta in developing human lung and other foregut derivatives. J Histochem Cytochem 46, 955-962.
Strutz, F., Okada, H., Lo, C., Danoff, T., Carone, R., Tomaszewski, J., and Neilson, E. (1995). Identification and characterization of a fibroblast marker-FSP1. J Cell Biol 130, 393-405.
Tissir, F., Bar, I., Jossin, Y., De Backer, O., and Goffinet, A. M. (2005). Protocadherin Celsr3 is crucial in axonal tract development. Nature neuroscience 8, 451-457.
Torres, M. A. (1996). Activities of the Wnt-1 class of secreted signaling factors are antagonized by the Wnt-5A class and by a dominant negative cadherin in early Xenopus development. The Journal of cell biology 133, 1123-1137.
Veeman, M. T., Slusarski, D. C., Kaykas, A., Louie, S. H., and Moon, R. T. (2003). Zebrafish Prickle, a Modulator ofNoncanonical Wnt/Fz Signaling, Regulates Gastrulation Movements. Current Biology 13, 680-685.
Vegas, A. J., Veiseh, O., Gurtler, M., Millman, J. R., Pagliuca, F. W., Bader, A. R., Doloff, J. C., Li, J., Chen, M., Olejnik, K., et al. (2016). Long-term glycemic control using polymer-encapsulated human stem cell-derived beta cells in immune-competent mice. Nature medicine 22, 306-311.
Wang, Y., Thekdi, N., Smallwood, P., Macke, J., and Nathans, J. (2002). Frizzled-3 is required for the development of major fiber tracts in the rostral CNS. J Neurosci 22, 8563-8573.
Yamaguchi, T., Dumont, D., Conlon, R., Breitman, M., and Rossant, J. (1993). <flk-1, an fit-related receptor tyrosine kinase is an early marker for endothelial cell precursors.pdf>. Development 118, 489-498.
Yang, D., Scavuzzo, M. A., Chmielowiec, J., Sharp, R., Bajic, A., and Borowiak, M. (2016). Enrichment of G2/M cell cycle phase in human pluripotent stem cells enhances HDR-mediated gene repair with customizable endonucleases. Sci Rep 6, 21264.
Yoshihara, E., Wei, Z., Lin, C. S., Fang, S., Ahmadian, M., Kida, Y., Tseng, T., Dai, Y., Yu, R. T., Liddle, C., et al. (2016). ERRgamma Is Required for the Metabolic Maturation of Therapeutically Functional Glucose-Responsive beta Cells. Cell metabolism 23, 622-634.
Yoshitomi, H., and Zaret, K. S. (2004). Endothelial cell interactions initiate dorsal pancreas development by selectively inducing the transcription factor Ptf1a. Development 131, 807-817.
Zaykov, A. N., Mayer, J. P., and DiMarchi, R. D. (2016). Pursuit of a perfect insulin. Nature reviews Drug discovery 15, 425-439.
Zhu, S., Russ, H. A., Wang, X., Zhang, M., Ma, T., Xu, T., Tang, S., Hebrok, M., and Ding, S. (2016). Human pancreatic beta-like cells converted from fibroblasts. Nature communications 7, 10080.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method of treating an individual for diabetes, one or more diabetes-related conditions, or pre-diabetes, comprising the step of administering to the individual an effective amount of insulin-producing cells produced upon exposure of insulin-lacking cells to one or more agents, wherein the one or more agents are selected from the group consisting of Wnt5a, FGF7, WNT3a, HGF, THBS2, IGF1, PDPN, LIF, endocan, SERPINF1, EGF, and a combination thereof.

2. The method of claim 1, wherein the insulin-lacking cells were stem cells, pluripotent cells, induced pluripotent stem cells, or a mixture thereof.

3. The method of claim 2, wherein the insulin-lacking cells are embryonic stem cells.

4. The method of claim 1, wherein the diabetes is type I or type II.

5. The method of claim 1, wherein the cells are autologous to the individual.

6. The method of claim 1, wherein the cells are allogeneic to the individual.

7. The method of claim 1, further comprising the step of obtaining the insulin-lacking cells from the individual.

8. The method of claim 1, further comprising the step of obtaining the insulin-lacking cells from a different individual.

9. The method of claim 1, wherein the individual is an infant, child, adolescent, or adult.

10. The method of claim 1, wherein the cells are administered to the individual by injection.

11. The method of claim 1, wherein the cells that are administered to the individual are encapsulated.

12. The method of claim 10, wherein the cells are injected into a portal vein connecting the liver and the pancreas.

13. The method of claim 1, wherein the cells are administered to the individual in an encapsulation device.

14. The method of claim 1, wherein the cells are administered to the individual in arginate bubbles.

15. The method of claim 1, wherein the cells are administered to the individual more than once.

16. The method of claim 1, wherein the insulin-producing cells or insulin-lacking cells are engineered to produce one or more non-endogenous gene products.

17. The method of claim 16, wherein one or more cell surface receptors in the cells are modified to avoid immune system recognition of the cells.

18. The method of claim 1, wherein the one or more agents comprise, consist of, or consist essentially of Endocan, SERPINF1, WNT5A, HGF, and a combination thereof.

19. The method of claim 1, wherein the one or more agents comprise, consist of, or consist essentially of Endocan and SERPINF1.

20. The method of claim 1, wherein the one or more agents comprise, consist of, or consist essentially of Endocan and WNT5A.

21. The method of any one of the preceding claims, wherein the one or more agents comprise, consist of, or consist essentially of Endocan and HGF.

22. The method of claim 1, wherein the one or more agents comprise, consist of, or consist essentially of SERPINF1 and WNT5A.

23. The method of claim 1, wherein the one or more agents comprise, consist of, or consist essentially of SERPINF1 and HGF.

24. The method of claim 1, wherein the one or more agents comprise, consist of, or consist essentially of WNT5A and HGF.

25. The method of claim 1, wherein the one or more agents comprise, consist of, or consist essentially of Endocan and SERPINF1 and WNT5A.

26. The method of claim 1, wherein the one or more agents comprise, consist of, or consist essentially of Endocan and SERPINF1 and HGF.


SEQUENCE LISTING (42 sequences)

SEQ ID NO: 1 - DNA, 20 bp, Artificial Sequence
  Synthetic Nucleotide
tgcaggctcagattcaggaa

SEQ ID NO: 2 - DNA, 20 bp, Artificial Sequence
  Synthetic Nucleotide
ctccggtactcagtggactc

SEQ ID NO: 3 - DNA, 20 bp, Artificial Sequence
  Synthetic Nucleotide
tcccctaagaattgctgcca

SEQ ID NO: 4 - DNA, 20 bp, Artificial Sequence
  Synthetic Nucleotide
ttcttcccaacacgccaatg

SEQ ID NO: 5 - DNA, 20 bp, Artificial Sequence
  Synthetic Nucleotide
aggggtgaagaagatgggtg

SEQ ID NO: 6 - DNA, 20 bp, Artificial Sequence
  Synthetic Nucleotide
ccagtcacaccagcaatcac

SEQ ID NO: 7 - DNA, 20 bp, Artificial Sequence
  Synthetic Nucleotide
ttacttgcaggggacagagg

SEQ ID NO: 8 - DNA, 20 bp, Artificial Sequence
  Synthetic Nucleotide
ttcccggtagaagcacttgt

SEQ ID NO: 9 - DNA, 20 bp, Artificial Sequence
  Synthetic Nucleotide
taccaggacgctttcaccat

SEQ ID NO: 10 - DNA, 20 bp, Artificial Sequence
  Synthetic Nucleotide
aaaggctgctggaaaatggg

SEQ ID NO: 11 - DNA, 20 bp, Artificial Sequence
  Synthetic Nucleotide
agagaccccgttgcctaaaa

SEQ ID NO: 12 - DNA, 20 bp, Artificial Sequence
  Synthetic Nucleotide
cagtacacggtgaggaaggt

SEQ ID NO: 13 - DNA, 20 bp, Artificial Sequence
  Synthetic Nucleotide
tgcaacacttgtgtctgtcg

SEQ ID NO: 14 - DNA, 20 bp, Artificial Sequence
  Synthetic Nucleotide
cgaaaggtcccagggttact

SEQ ID NO: 15 - DNA, 20 bp, Artificial Sequence
  Synthetic Nucleotide
agcctttgtgaaccaacacc

SEQ ID NO: 16 - DNA, 20 bp, Artificial Sequence
  Synthetic Nucleotide
gctggtagagggagcagatg

SEQ ID NO: 17 - DNA, 21 bp, Artificial Sequence
  Synthetic Nucleotide
aagtctaccaaagctcacgcg

SEQ ID NO: 18 - DNA, 15 bp, Artificial Sequence
  Synthetic Nucleotide
gtaggcgccgcctgc

SEQ ID NO: 19 - DNA, 17 bp, Artificial Sequence
  Synthetic Nucleotide
ctccgctcggattcctc

SEQ ID NO: 20 - DNA, 20 bp, Artificial Sequence
  Synthetic Nucleotide
caaagcaactcctgggctta

SEQ ID NO: 21 - DNA, 20 bp, Artificial Sequence
  Synthetic Nucleotide
tgtgcacaggagccaagagt

SEQ ID NO: 22 - DNA, 21 bp, Artificial Sequence
  Synthetic Nucleotide
attttcttgctgccagtctgg

SEQ ID NO: 23 - DNA, 24 bp, Artificial Sequence
  Synthetic Nucleotide
aagcatttactttgtggctggatt

SEQ ID NO: 24 - DNA, 25 bp, Artificial Sequence
  Synthetic Nucleotide
tgatctggatttctcctctgtgtct

SEQ ID NO: 25 - DNA, 22 bp, Artificial Sequence
  Synthetic Nucleotide
cagaaatacagtgtggcagaca

SEQ ID NO: 26 - DNA, 18 bp, Artificial Sequence
  Synthetic Nucleotide
acacggttcgcagctttc

SEQ ID NO: 27 - DNA, 20 bp, Artificial Sequence
  Synthetic Nucleotide
ctcctagcaggacttggcat

SEQ ID NO: 28 - DNA, 20 bp, Artificial Sequence
  Synthetic Nucleotide
tggctgtcaagaatcatgga

SEQ ID NO: 29 - DNA, 18 bp, Artificial Sequence
  Synthetic Nucleotide
tgcaggaagcatgagctg

SEQ ID NO: 30 - DNA, 18 bp, Artificial Sequence
  Synthetic Nucleotide
gtgcgttgagtgggaagg

SEQ ID NO: 31 - DNA, 20 bp, Artificial Sequence
  Synthetic Nucleotide
ggacaggagagaatgggaca

SEQ ID NO: 32 - DNA, 20 bp, Artificial Sequence
  Synthetic Nucleotide
tgtgtttgagggtgtgcagt

SEQ ID NO: 33 - DNA, 20 bp, Artificial Sequence
  Synthetic Nucleotide
tgccaactatgagagccatc

SEQ ID NO: 34 - DNA, 20 bp, Artificial Sequence
  Synthetic Nucleotide
caacgtggatacaagaacgc

SEQ ID NO: 35 - DNA, 20 bp, Artificial Sequence
  Synthetic Nucleotide
tttttgggtgtgttgcctct

SEQ ID NO: 36 - DNA, 19 bp, Artificial Sequence
  Synthetic Nucleotide
agaccttccggaggatgtg

SEQ ID NO: 37 - DNA, 20 bp, Artificial Sequence
  Synthetic Nucleotide
tttcggtcaaatccttcctg

SEQ ID NO: 38 - DNA, 20 bp, Artificial Sequence
  Synthetic Nucleotide
tgcaaaggccaagagaagac

SEQ ID NO: 39 - DNA, 19 bp, Artificial Sequence
  Synthetic Nucleotide
gtggtccttcttgtgctgc

SEQ ID NO: 40 - DNA, 19 bp, Artificial Sequence
  Synthetic Nucleotide
gtacccgcacttgcacaac

SEQ ID NO: 41 - DNA, 20 bp, Artificial Sequence
  Synthetic Nucleotide
catgttgctcacggaggagt

SEQ ID NO: 42 - DNA, 20 bp, Artificial Sequence
  Synthetic Nucleotide
tttaaactgccatgcactcg